(19) United States
(12) Patent Application Publication
(10) Pub. No.: US 2019/0228852 A1
(43) Pub. Date: Jul. 25, 2019

(54) Automated Medication Adherence System

(71) Applicant: itracHealth Corp., Canoga Park, CA (US)

(72) Inventors: César Manuel García, North Miami, FL (US); Bart Jan Wanders, Coto De Caza, CA (US); Philip Eugene Alei, Carlsbad, CA (US); Conrado Orlando Diaz, Camarillo, CA (US)

(21) Appl. No.: 16/373,382

(22) Filed: Apr. 2, 2019

Related U.S. Application Data
(63) Continuation-in-part of application No. 15/800,318, filed on Nov. 1, 2017, now abandoned.
Application No. 15/800,318 is a continuation of application No. 15/073,096, filed on Mar. 17, 2016, now Pat. No. 9,836,583.

(51) Int. Cl. G16H 20/13; G07F 17/00; A61J 7/04; A61J 7/00; G07F 11/54

(52) U.S. Cl. CPC G16H 20/13; G07F 17/0092; A61J 7/0481; A61J 2200/30; G07F 11/54; A61J 2205/10; A61J 7/0084

(57) ABSTRACT

An automated medication adherence system comprising: a reservoir; a dispensing tray; and a plurality of sensors. The reservoir may comprise two vertically stacked successive stages, an upper first stage and a lower second stage, a central agitation stalk, an outer wall cylinder, an inner wall cylinder, and one or more actuators. The reservoir may receive, store, and dispense a plurality of pills. The central agitation stalk may be contained and rotatable within the inner wall cylinder. The pills may be transferred from the first stage to the second stage one pill at a time and then transferred to the dispenser tray one pill at a time.

### CROSS-REFERENCE TO RELATED APPLICATIONS

This U.S. Non-Provisional patent application is a Continuation in Part of U.S. Non-Provisional patent application Ser. No. 15/800,318, filed on Nov. 1, 2017, which is a Continuation Application of U.S. Non-Provisional patent application Ser. No. 15/073,096, filed on Mar. 17, 2016, now U.S. Pat. No. 9,836,583, the contents of which both of which expressly incorporated herein by this reference as though set forth in their entirety

### FIELD OF USE

The present disclosure relates generally to the field of article dispensing, and more specifically, to an automated medication adherence system for use in user's home.

### BACKGROUND

In 2010, there were 15.4 million Medicare beneficiaries over the age of 65 with four or more chronic conditions, of which 43% were hospitalized at least once during the year. Further, an estimated 39% of the elderly population has some type of disability (i.e., difficulty in hearing, vision, cognition, ambulation, self-care, or independent living). For a considerable number of individuals, these types of disabilities may make it difficult to adhere to a medication schedule.

Many of these individuals may depend on untrained volunteers, family, or friends to help them manage their medications. Current estimates state there are 34.2 million Americans that have provided unpaid care to an adult 50 years old or over. Approximately a quarter of these caregivers provide 41 or more hours of care per week, typically for a close relative who has been hospitalized in the past year. These caregivers often experience stress, physical and financial strain, and adverse impacts on their health while they perform complex medical and nursing responsibilities.

Furthermore, the U.S. Census Bureau anticipates population growth of 60% for the age group over 65 years old and a population decline of 1% in the age group 45 to 64 years old between 2014 and 2030. This major demographic shift may significantly affect the support system for the elderly. Even individuals that receive assistance from a paid home health aide may still have issues managing their medications, as the majority of home health aides do not administer medication or provide assistance with self-administration of medications. Many of these home health aides are prohibited to administer medication by state law or have not obtained the required medication technician certification required by most nurse delegation programs due to cost and potential liability concerns.

Even though the elderly currently comprise only 12% of the population, they consume 33% of all prescription drugs with two out of five Medicare beneficiaries taking five or more prescription medications. The large number of medications prescribed to the elderly and chronically ill, combined with the cognitive and sometimes physical challenges of following multiple medication regimens, reduce a patient's ability to fully benefit from prescribed medications. It has been estimated that 20-30% of medication prescriptions are never filled and 50% of the time medication is not continued and completed as prescribed. Polypharmacy, defined as taking multiple medications concurrently to treat coexisting diseases, with the elderly typically leads to medication non-adherence and is estimated to occur among 25%-75% of elderly patients, with the rate of occurrence increasing in proportion to the number of drugs and daily dosages prescribed. Lack of medication adherence can result in disease progression, death and higher costs to the healthcare system. Furthermore, non-adherence was estimated to account for 10% of hospital admissions and 23% of nursing home admissions. The New England Healthcare Institute calculates non-adherence along with suboptimal prescribing, drug administration and diagnosis could result in up to $290 billion in losses annually in the US. Additionally, estimates report the effect of poor medication adherence results in approximately 125,000 deaths in the US annually.

There are a growing number of studies that have documented net savings associated with higher medication adherence across a range of common chronic conditions. One study demonstrated improved medication adherence might provide a net economic return for certain chronic conditions, including diabetes, hypertension, hypercholesterolemia and congestive heart failure. Consequently, the study noted increased adherence to drug therapy reduced a patient's need for medical services, including hospitalizations and emergency room visits.

However, independent management of drug administration is a relatively ineffective way to increase medication adherence. Seven-day pillboxes are probably the most common products used, but they require manual sorting of pills on a weekly basis. This is an unreliable and cumbersome process that sometimes requires assistance from a caregiver or pharmacist. One study noted that the majority of elderly patients may be unable to open and access their medications from multi-compartment pillboxes with ease, and cognitively impaired patients may experience even more difficulties than others. Forgetfulness is a major factor contributing to non-adherence, with an estimated 30% of patients with chronic conditions asserting forgetfulness. This poses a further challenge to independent seniors, which are at a higher risk of forgetting to take their medication if they experience increased busyness. However, most pillboxes do not provide interactive reminders or instructions, and are thus inadequate solutions in this respect. Another downside to pillboxes is that they may promote cross-contamination, as different pills are placed inside a small compartment together.

Smart phone applications have been developed to assist in medication adherence through reminders and alerts, but are not comprehensive solutions addressing the specific needs for patients with several chronic conditions and potentially suffering from physical and cognitive impairments. As a result, reminders and alarms alone are not likely to improve adherence unless they are designed to provide relevant information with interactive features to facilitate addressing these concerns on a timely basis. Lastly, there are mail order pharmacies that specialize in pre-sorting prescription pills into pill pouches or blister packs and shipping directly to patients. However, the process of managing medication changes is cumbersome and apt to wasting a supply of medication. Although the pre-sorted packets help to simplify the medication administration, this is clearly not an interactive system with real-time capability to remind, instruct, monitor, and alert the status of the patient's medication adherence record.

Currently, there are no medication adherence solutions on the market that are comprehensive, fully automated, and requires no programming by the user. In addition, critical information such as medication formularies, e-prescriptions and pharmaceutical databases are kept in “silos” and are not readily available in an integrated fashion, making it difficult to retrieve data for contextual analysis. Consequently, even the more advanced medication administration products on the market still require manual pill sorting and programming of alarms and reminders
a challenging task for this at-risk population.

Accordingly, there exists a need for a device that provides an effective solution for both patients and health care providers regarding the patient's adherence or compliance with complicated medication regimens. In particular, there exists a need for an automated medication adherence system to help organize the dispensing of many different sizes and shapes of pills and capable of managing a schedule of different pills to be taken at different scheduled times. Such a system should enhance the interaction between the patient and health care provider by allowing the health care provider to be alerted when the patient is not taking the medication according to the medication schedule.

### SUMMARY

To minimize the limitations in the cited references, and to minimize other limitations that will become apparent upon reading and understanding the present specification, the present specification discloses a new and improved automated medication adherence system.

One embodiment may be an automated medication adherence system, comprising a housing and an electronic interface. The electronic interface may use such data to automatically program the control logic for operating the electromechanical operation of the adherence system and the medication dispensing and scheduling functions using information from pharmacy prescription records, pharmaceutical databases and medical professionals. The electronic interface acquires data to schedule, alert and record therapies for pill and non-pill medications. The housing may comprise a medication dispensing and lock-out module. The housing may be configured to contain a plurality of reservoirs wherein the plurality of reservoirs may be configured for receiving, storing, and dispensing one or more medications. The housing may comprise an access cover configured to have closed and opened position. The plurality of reservoirs may be accessible when the access cover is in the opened position. The access cover may comprise a pill loading assembly wherein the pill loading assembly may be configured to allow one or more medications to be loaded into at least one of the plurality of reservoirs. The electronic interface may comprise a computing component and multiple display components. The electronic interface may be on an exterior portion of the housing. The electronic interface may be programmable, such that the electronic interface accepts data relating to one or more medications from pharmacies, medical professionals, database companies and other authorized users. Each of the plurality of reservoirs may be configured to receive, store and dispense a homogenous type of medication from the one or more medications. Each of the plurality of reservoirs may comprise one or more sensors and two successive stages, a first stage and a second stage. The one or more medications may be moved from the first stage to the second stage and then from the second stage to the pill delivery and lock-out module. The one or more medications may be a plurality of pills. The one or more sensors may be configured to determine when a single pill of the plurality of pills passes through each of the two successive stages and may control pill ejection from stage 1 or 2, as appropriate. The automated medication adherence system may further comprise a rotating carrier configured to engage with the plurality of reservoirs, such that the plurality of reservoirs may be configured to rotate within the housing. Although the carrier shown is configured to move in a rotational manner, the carrier may move or slide in any manner, vertically, horizontally, and the like, so long as the reservoirs are moved to and from a position of interacting with the dispensing tray.

The electronic interface may rotate the plurality of reservoirs in response to the data relating to the one or more medications. The data relating to the one or more medications may be selected from the group of data gathered or created through manipulation of data from prescription records, pharmaceutical databases and proprietary data bases consisting of information such as: a pill identity; a user identity; a dosage schedule; medication format (pill or non-pill; such as inhalers, solutions, creams, etc.), pill images, pharmaceutical indications for use, instructions (directions) for use, physical and chemical description of the medications, refills, side effect information and other information customarily used to manage and administer medications. The two successive stages may be configured to be stacked, such that the first stage may be substantially above the second stage. Each of the two successive stages may comprise a through-hole, such that there are two through-holes, a first through-hole and a second through-hole. The first through-hole and the second through-hole may be selectively openable and closable in response to the electronic interface. Each of the two successive stages may receive and dispense the one or more medications through the two through-holes. Each of the plurality of reservoirs may comprise a central agitation stalk, an outer wall cylinder; an inner wall cylinder; optionally one or more actuators and sensors. The central agitation stalk may be configured to be substantially contained within the inner wall cylinder, and the inner wall cylinder may be configured to be substantially contained within the outer wall cylinder. The central agitation stalk may be configured to be rotatable within the inner wall cylinder. The central agitation stalk may comprise a fin portion, a wave surface, and a ribbed cone surface. The fin portion may comprise a plurality of fins that may be configured to prevent the one or more medications from clumping together. The wave surface may be a base of the first stage; and the ribbed cone surface may be a base of the second stage. The ribbed cone may have ribs, undercuts, channels or any type of texture or geometry suitably to transport the pills to the stage 2 through-hole. A combination of wave surface and the ribbed cone can be used at both stage 1 and stage 2. The one or more actuators may be configured to rotate and agitate the central agitation stalk and at least one of the inner wall cylinder and the outer wall cylinder. The outer wall cylinder may comprise one or more outer wall cylinder openings and one or more chutes. The inner wall cylinder may comprise one or more inner wall cylinder openings. At least one of the one or more actuators may be configured to rotate at least one of the inner wall cylinder and the outer wall cylinder, such that the inner wall cylinder and the outer wall cylinder may be rotated with respect to each other. When the inner wall cylinder and the outer wall cylinder are rotated with respect to each other, the one or more outer wall cylinder openings and the one or more inner wall cylinder openings may align to form the two through-holes that best match the solid geometry of the pill in that reservoir. The computing component may comprise one or more logic algorithms. The one or more sensors, the one or more actuators, and the one or more logic algorithms may be configured to control the inner wall cylinder, the outer wall cylinder, and the central agitation stalk to ensure that the one or more medications may be transferred, one pill at a time, from the first stage to the second stage. The one or more sensors, the one or more actuators, and the one or more logic algorithms may be configured to control the inner wall cylinder, the outer wall cylinder, and the central agitation stalk to ensure that the one or more medications may be transferred, one pill at a time, from the second stage to the pill delivery and lock-out module. The electronic interface may alert a user when the one or more medications are dispensed, such that a dispensed medication may be created. The electronic interface may also alert a user when the one or more non-pill medications are required. The electronic interface may alert the user, one or more authorized individuals, and/or one or more health care providers when a user has not removed the dispensed medication from the pill delivery and lock-out module in accordance with the data relating to the one or more medications. The pill delivery and lock-out module may comprise three functions created by the translation of a pill transporter. The pill transporter may create a holding tray, a dispensing tray and a lock-out tray depending on the position of the pill transporter. If the pill transporter is in the neutral position, below the reservoir, it holds the pill or plurality of pills dispensed creating a holding tray. The transporter moves forward to create and form a pill dispensing tray that opens toward the user when the user is ready to take the pills. If the pills are not removed by the patient after a predetermined or calculated amount of time, or erroneously dispensed, the pill transporter may move in reverse to transfer the pills to a lock-out tray.

Another embodiment of the automated medication adherence system may comprise: a housing; an electronic interface; and a rotating carrier. The housing may comprise a pill loading assembly and a pill delivery and lock-out module. The housing may be configured to contain a plurality of reservoirs. The plurality of reservoirs may be configured for receiving, storing, and dispensing one or more medications. The rotating carrier may be configured to engage with the plurality of reservoirs, such that the plurality of reservoirs may be configured to rotate within the housing. The housing may comprise an access cover configured to have a closed and an opened position typically used for set-up and maintenance. The housing may comprise a reservoir loading door configured to have a closed and an opened position to be accessed by the end user. The plurality of reservoirs may be accessible when the access cover or the reservoir loading door are in the opened position. The reservoir loading door may comprise a reservoir loading assembly configured to allow the plurality of reservoirs to be loaded. The electronic interface may comprise a computing component and one or more display components. The user interface portion of the electronic interface may be on an exterior portion of the housing. The electronic interface may be manually programmed by the user or automatically by accepting data relating to the one or more medications, prescriptions, and prescription processes (e.g. refills, medication changes). The data relating to the one or more medications may be selected from the group of data from pharmacy prescription records, providers prescription records, pharmaceutical databases or proprietary databases consisting of: a pill identity; a user identity; a dosage schedule; medication format (pill or non-pill; such as inhalers, solutions, creams, etc.), pharmaceutical indications for use, instructions (directions) for use, physical and chemical description of the medications, pill images, instruction for use, refills, side effect information and other information customarily use to manage and administer medications. The electronic interface may rotate the plurality of reservoirs in response to the data relating to the one or more medications. Pills may be loaded through the pill loading assembly. Each of the plurality of reservoirs may be configured to receive, store and dispense a homogenous type of the medication. Each of the plurality of reservoirs may comprise one or more sensors and two successive stages, a first stage and a second stage. The medication may be moved from the first stage to the second stage and then from the second stage to the pill delivery and lock-out module. The two successive stages may be configured to be stacked, such that the first stage may be substantially above the second stage. Each of the two successive stages may comprise a through-hole, such that there may be two through-holes, a first through-hole and a second through-hole. The first through-hole and the second through-hole may be selectively openable and closable in response to the electronic interface. Each of the two successive stages may receive and dispense the medication through the two through-holes. The medication may be a plurality of pills. The one or more sensors may be configured to determine when a single pill of the plurality of pills passes through each of the two through-holes and triggers the immediate closing of the through-hole. Each of the plurality of reservoirs may comprise a central agitation stalk, an outer wall cylinder, an inner wall cylinder, and one or more actuators. The central agitation stalk may be configured to be substantially contained within the inner wall cylinder, and wherein the inner wall cylinder may be configured to be substantially contained within the outer wall cylinder. The central agitation stalk may be configured to be rotatable within the inner wall cylinder. The central agitation stalk may comprise a fin portion, a wave surface, and a ribbed cone surface. The fin portion may comprise a plurality of fins that may be configured to prevent the medication from clumping together. The wave surface may be a base of the first stage. The ribbed cone surface may be a base of the second stage. The central agitation stalk may be configured to engage with at least one of the one or more actuators in order to be rotated. The central agitation stalk may comprise a plurality of gear teeth, which may be configured to be engaged with at least one of the one or more actuators. The one or more actuators may be configured to rotate the central agitation stalk and at least one of the inner wall cylinder and the outer wall cylinder. The one or more actuators may be configured to rotate or agitate the central agitation stalk and at least one of the inner wall cylinder and the outer wall cylinder. The outer wall cylinder may comprise one or more outer wall cylinder openings and one or more chutes. The inner wall cylinder may comprise one or more inner wall cylinder openings. At least one of the one or more actuators may be configured to rotate at least one of the inner wall cylinder and the outer wall cylinder, such that the inner wall cylinder and the outer wall cylinder may be rotated with respect to each other. When the inner wall cylinder and the outer wall cylinder are rotated with respect to each other, the one or more outer wall cylinder openings and the one or more inner wall cylinder openings may align to form the two through-holes. The computing component may comprise one or more logic algorithms. The one or more sensors, the one or more actuators, and the one or more logic algorithms may be configured to control the inner wall cylinder, the outer wall cylinder, and the central agitation stalk to ensure that the medication may be transferred, one pill at a time, from the first stage to the second stage. The one or more sensors, the one or more actuators, and the one or more logic algorithms may be configured to control the inner wall cylinder, the outer wall cylinder, and the central agitation stalk to ensure that the medication may be transferred, one pill at a time, from the second stage to the pill delivery and lock-out module. The one or more logic algorithms may be configured to schedule and control the dispensing of medication according to the corresponding prescription or plurality of prescriptions and instructions for use.

Another embodiment of the medication reservoir for an automated medication adherence system may comprise two successive stages, a first stage and a second stage. The reservoir may be configured for receiving, storing, and dispensing a plurality of pills. Dispensing of the plurality of pills by the reservoir may be controlled by an electronic interface. The plurality of pills may be moved from the first stage to the second stage one pill at a time. The plurality of pills may be moved from the second stage to a pill delivery and lock-out module one pill at a time. Each of the two successive stages may comprise a through-hole, such that there may be two through-holes, a first through-hole and a second through-hole. The medication reservoir may further comprise one or more sensors wherein the one or more sensors may be configured to determine when a single pill of the plurality of pills passes through each of the two through-holes. The first and second through-holes may be selectively openable and closable in response to the electronic interface. The two successive stages may be configured to be stacked, such that the first stage may be substantially above the second stage. The reservoir may further comprise a central agitation stalk, an outer wall cylinder, an inner wall cylinder, and one or more actuators. The central agitation stalk may be configured to be substantially contained within the inner wall cylinder, and the inner wall cylinder may be configured to be substantially contained within the outer wall cylinder. The central agitation stalk may be configured to be rotatable within the inner wall cylinder. The central agitation stalk may comprise a fin portion, a wave surface, and a ribbed cone surface. The fin portion may comprise a plurality of fins that may be configured to prevent the one or more medications from clumping together. The wave surface may be a base of the first stage and the ribbed cone surface may be a base of the second stage. The one or more actuators may be configured to rotate and agitate the central agitation stalk and at least one of the inner wall cylinder and the outer wall cylinder. The outer wall cylinder may comprise one or more outer wall cylinder openings and one or more chutes. The inner wall cylinder may comprise one or more inner wall cylinder openings. At least one of the one or more actuators may be configured to rotate at least one of the inner wall cylinder and the outer wall cylinder, such that the inner wall cylinder and the outer wall cylinder may be rotated with respect to each other. The inner wall cylinder and the outer wall cylinder may be rotated with respect to each other, the one or more outer wall cylinder openings and the one or more inner wall cylinder openings may align to form the two openings. The electronic interface may comprise one or more logic algorithms. The one or more sensors, the one or more actuators, and the one or more logic algorithms may be configured to control the inner wall cylinder, the outer wall cylinder, and the central agitation stalk to ensure that the one or more medications may be transferred, one pill at a time, from the first stage to the second stage. The one or more sensors, the one or more actuators, and the one or more logic algorithms may be configured to control the inner wall cylinder, the outer wall cylinder, and the central agitation stalk to ensure that the one or more medications may be transferred, one pill at a time, from the second stage to the pill delivery and lock-out module.

One embodiment may be an automated medication adherence system, comprising: a housing; one or more sensors; wherein the housing comprises a pill delivery and lock-out module; wherein the housing is configured to contain a plurality of reservoirs; wherein the plurality of reservoirs are configured for receiving, storing, and dispensing a plurality of pills; wherein the housing comprises an access cover; wherein the housing comprises a reservoir loading door; wherein the access cover comprises a pill loading assembly; wherein the pill loading assembly is configured to allow the plurality of pills to be loaded into at least one of the plurality of reservoirs; wherein the reservoir loading door comprises a reservoir loading assembly; wherein the reservoir loading door is configured to allow the one or more reservoirs to be loaded and locked into a rotating carrier; wherein each of the plurality of reservoirs is configured to receive, store and dispense a homogenous type of pills of the plurality of pills; wherein each of the plurality of reservoirs comprises a first stage, a second stage, a central agitation stalk, an outer wall cylinder, an inner wall cylinder, a homing strip, and one or more actuators; wherein the central agitation stalk is configured to be substantially contained within the inner wall cylinder, and wherein the inner wall cylinder is configured to be substantially contained within the outer wall cylinder; wherein the central agitation stalk is configured to be rotatable within the inner wall cylinder; wherein the one or more sensors comprise one or more optical sensors, such that at least one of the one or more optical sensors collects data regarding rotational location of the homing strip, wherein the rotational location of the homing strip is usable to determine a rotational configuration of the reservoir; wherein the central agitation stalk comprises a fin portion, a wave surface, and a ribbed cone surface; wherein the fin portion comprises a plurality of fins that are configured to prevent the plurality of pills from clumping together; wherein the wave surface is a base of the first stage; wherein the ribbed cone surface is a base of the second stage; and wherein the wave surface is configured to cause the pills to travel from the first stage to the second stage and the ribbed cone surface is configured to cause the pills to travel from the second stage to the pill delivery and lock-out module. Each of one or more sensors may be connected with a computing component. The one or more sensors may be configured to determine when a single pill of the plurality of pills passes through each of the two successive stages. The sensor may also determine when more than one pill has slipped through. In one embodiment at least one of the one or more sensors is a light curtain sensor. The automated medication adherence system may also include a rotating carrier; wherein the rotating carrier may be configured to engage with the plurality of reservoirs, such that the plurality of reservoirs are configured to rotate within the housing. The homing strip may comprise a pattern, or alternatively, a magnetic target. The computing component may cause the plurality of reservoirs to rotate in response to the data regarding rotational location of the homing strip. The data relating to the plurality of pills may be used to determine appropriate settings for dispensing the plurality of pills. The data relating to the plurality of pills may be selected from the group of data consisting of: a pill identity; a user identity; a dosage schedule; a medication format; a pill image; a pill geometry; a plurality of pharmaceutical indications for use; instructions for use; a physical description; a chemical description; a refill information; and/or a plurality of side effect information. The first stage and the second stage are configured to be stacked, such that the first stage is physically above the second stage; wherein each of the first stage and the second stage may comprise a through-hole, such that there are two through-holes, a first through-hole and a second through-hole. The first through-hole and the second through-hole may be continuously variable such that the rotation of the plurality of reservoirs allows pills of different sizes to pass through the first through-hole and the second through-hole. The first through-hole and the second through-hole may be selectively openable and closable in response to the computing component; wherein each of the first through-hole and the second through-hole, when open, may allow a plurality of pills to pass.

Another embodiment may be an automated medication adherence system, comprising: a reservoir; a dispensing tray; and a plurality of sensors, comprising at least a first through-hole sensor, a second through-hole sensor, and a dispensing tray sensor; wherein the reservoir comprises two vertically stacked successive stages, an upper first stage and a lower second stage, a central agitation stalk, an outer wall cylinder, an inner wall cylinder, and one or more actuators; wherein the reservoir is configured for receiving, storing, and dispensing a plurality of pills; wherein the central agitation stalk is configured to be substantially contained within the inner wall cylinder, and wherein the inner wall cylinder is configured to be substantially contained within the outer wall cylinder; and wherein the central agitation stalk is configured to be rotatable within the inner wall cylinder; wherein the central agitation stalk comprises a fin portion, a wave surface, and a ribbed cone surface; wherein the fin portion comprises a plurality of fins that are configured to prevent the plurality of pills from clumping together; wherein the wave surface is a base of the first stage; wherein the ribbed cone surface is a base of the second stage; wherein dispensing of the plurality of pills by the reservoir is controlled by an electronic interface based on data gathered by the plurality of sensors; wherein one or more of the plurality of pills are transferred from the first stage to the second stage one pill at a time; wherein the one or more of the plurality of pills that are transferred to the second stage are transferred from the second stage to the dispenser tray one pill at a time. Each of the first stage and the second stage may comprise a through-hole, such that there are two through-holes, a first through-hole and a second through-hole. The first through-hole sensor may determine when a single pill of the plurality of pills passes through the first through-hole; and wherein the second through-hole sensor may determine when a single pill of the plurality of pills passes through the second through-hole. The first through-hole and second through-hole may be selectively openable and closable in response to the electronic interface. The first through-hole and the second through-hole may be variable in size such that rotation of the inner wall cylinder and the outer wall cylinder with respect to each other allows pills of different sizes to pass through the first through-hole and the second through-hole. The one or more of the plurality of pills transferred to the tray dispenser from the second stage may be sensed by the dispenser tray sensor before being dispensed. The dispenser tray sensor may determine whether the one or more of the plurality of pills dispensed in the tray dispenser are removed appropriately; wherein all of the plurality of pills not removed appropriately from the dispenser tray may be removed by the system from the dispenser tray to prevent over dosing.

Another embodiment may be an automated medication adherence system, comprising: a reservoir; a dispensing tray; and a plurality of sensors, comprising at least a first through-hole sensor, a second through-hole sensor, and a dispensing tray sensor; wherein the reservoir comprises two vertically stacked successive stages, an upper first stage and a lower second stage, a central agitation stalk, an outer wall cylinder, an inner wall cylinder, and one or more actuators; wherein the reservoir is configured for receiving, storing, and dispensing a plurality of pills; wherein the central agitation stalk is configured to be substantially contained within the inner wall cylinder, and wherein the inner wall cylinder is configured to be substantially contained within the outer wall cylinder; wherein the central agitation stalk is configured to be rotatable within the inner wall cylinder; wherein the central agitation stalk comprises a fin portion, a wave surface, and a ribbed cone surface; wherein the fin portion comprises a plurality of fins that are configured to prevent the plurality of pills from clumping together; wherein the wave surface is a base of the first stage; wherein the ribbed cone surface is a base of the second stage; wherein dispensing of the plurality of pills by the reservoir is controlled by an electronic interface based on data gathered by the plurality of sensors; wherein one or more of the plurality of pills are transferred from the first stage to the second stage one pill at a time; wherein the one or more of the plurality of pills that are transferred to the second stage are transferred from the second stage to the dispenser tray one pill at a time; wherein the one or more actuators are configured to rotate and agitate the central agitation stalk and rotate at least one of the inner wall cylinder and the outer wall cylinder, such that the inner wall cylinder and the outer wall cylinder are rotated with respect to each other; wherein the outer wall cylinder comprises one or more outer wall cylinder openings and one or more chutes; wherein the inner wall cylinder comprises one or more inner wall cylinder openings; wherein when the inner wall cylinder and the outer wall cylinder are rotated with respect to each other, the one or more outer wall cylinder openings and the one or more inner wall cylinder openings align to form the two through-holes, a first through-hole and a second through-hole; wherein the electronic interface comprises one or more logic algorithms; wherein the one or more sensors, the one or more actuators, and the one or more logic algorithms are configured to control the inner wall cylinder, the outer wall cylinder, and the central agitation stalk to ensure that the plurality of pills are transferred, one pill at a time, from the first stage to the second stage; and wherein the one or more sensors, the one or more actuators, and the one or more logic algorithms are configured to control the inner wall cylinder, the outer wall cylinder, and the central agitation stalk to ensure that the plurality of pills are transferred, one pill at a time, from the second stage to the dispenser tray.

It is an object to provide an automated medication adherence system to schedule medication dosage, medication replenishment, medication stoppage, and treatment changeovers with minimal user intervention.

It is an object to provide an automated medication adherence system with the ability to dispense a prescribed medication with an accuracy of up to 1:100,000.

It is an object to provide an automated medication adherence system to safely manage frequent changes in medication treatment and many different sizes of pills.

It is an object to provide an automated medication adherence system to provide error-free medication loading by a patient with potential physical and cognitive limitations.

It is an object to provide an automated medication adherence system with mechanical pill handling that does not affect the integrity of the medication.

It is an object to provide an automated medication adherence system capable of acquiring and communicating prescription instructions.

It is an object to provide ease of use by enabling a system that uses a single type of reservoir that can be programmed to handle pills of all sizes, solid geometries and construction methods. The reservoir can be used at any position within the rotating carrier.

It is an object to provide and record “pro re nata” (on demand or as needed) medication events to patients, if required.

It is an objective to provide maximum patient safety avoiding over-dosage or wrong dosage or wrong medication).

It is an object to overcome the limitations of the prior art.

Other features and advantages will become apparent to those skilled in the art from the following detailed description and its accompanying drawings.

### BRIEF DESCRIPTION OF THE DRAWINGS

The drawings show illustrative embodiments, but do not depict all embodiments. Other embodiments may be used in addition to or instead of the illustrative embodiments. Details that may be apparent or unnecessary may be omitted for the purpose of saving space or for more effective illustrations. Some embodiments may be practiced with additional components or steps and/or without some or all components or steps provided in the illustrations. When different drawings contain the same numeral, that numeral refers to the same or similar components or steps.

FIG. 1 is an illustration of a perspective view of one embodiment of the automated medication adherence system.

FIG. 2 is an illustration of a perspective view of one embodiment of a central agitation stalk.

FIG. 3 is an illustration of a cross-section view of one embodiment of a reservoir that is contained within the automated medication adherence system.

FIG. 4 is an illustration of a perspective view of one embodiment of a reservoir that is contained within the automated medication adherence system.

FIG. 5 is an illustration of an exploded view of one embodiment of a reservoir that is contained within the automated medication adherence system.

FIG. 6 is an illustration of a cross-section view of one embodiment of the interior of the automated medication adherence system and shows the sensors and actuators.

FIG. 7 is an illustration of a close-up view of one embodiment of the second stage of a reservoir that is contained within the automated medication adherence system.

FIG. 8 is an illustration of a close-up view of one embodiment of a reservoir that is contained within the automated medication adherence system with continuously variable through-holes to best match the solid geometry of the pill loaded into a specific reservoir.

FIG. 9A is an illustration of a close-up view of one embodiment of the pill delivery and lock-out module in its neutral position that is contained within the automated medication adherence system.

FIG. 9B is an illustration of a close-up view of one embodiment of the pill delivery and lock-out module in its forward position that is contained within the automated medication adherence system.

FIG. 9C is an illustration of one embodiment of the pill delivery and lock-out module in its reverse (lock-out tray) position that is contained within the automated medication adherence system.

FIG. 10 is an illustration of one embodiment of the reservoir cover mechanism that opens and closes a reservoir fill opening that is contained within the automated medication adherence system.

FIG. 11 is an illustration of one embodiment of the reservoir loading door and the pill loading assembly that are contained within the automated medication adherence system.

FIG. 12 is a flow block diagram of one embodiment of the method of medication moving through the first stage of the automated medication adherence system.

FIG. 13 is a flow block diagram of one embodiment of the method of medication moving through Stage 2 of the automated medication adherence system.

FIG. 14 is a flow block diagram of one embodiment of the method of taking medication on an as-needed or away from home basis.

FIG. 15 is a flow block diagram of one embodiment of the method of programming the reservoirs and dispense logic using one or more algorithms using the computing component.

FIG. 16 is a flow block diagram of one embodiment of a method of dispensing medication using the automated medication adherence system.

FIG. 17 is an illustration of one embodiment of a homing strip.

FIG. 18 is an illustration of one embodiment of the dispensing tray showing that pills have been dispensed into the dispensing tray.

FIG. 19 is an illustration of one embodiment of the dispensing tray showing that a sensor determines whether the pills have been dispensed into the dispensing tray.

FIG. 20 is an illustration of one embodiment of the automated medication adherence system with the dispensing tray providing access to the pills.

FIG. 21 is an illustration of one embodiment of the dispensing tray showing that pills were not removed from the dispensing tray.

FIG. 22 is an illustration of one embodiment of the dispensing tray showing that pills can be removed from the dispensing tray by the automated medication adherence system.

### DETAILED DESCRIPTION OF ILLUSTRATIVE EMBODIMENTS

In the following detailed description of various embodiments, numerous specific details are set forth in order to provide a thorough understanding of various aspects of the embodiments. However, the embodiments may be practiced without some or all of these specific details. In other instances, well-known procedures and/or components have not been described in detail so as not to unnecessarily obscure aspects of the embodiments.

While some embodiments are disclosed here, other embodiments will become obvious to those skilled in the art as a result of the following detailed description. These embodiments are capable of modifications of various obvious aspects, all without departing from the spirit and scope of protection. The Figures, and their detailed descriptions, are to be regarded as illustrative in nature and not restrictive. Also, the reference or non-reference to a particular embodiment shall not be interpreted to limit the scope of protection.

In the following description, certain terminology is used to describe certain features of one or more embodiments. For purposes of the specification, unless otherwise specified, the term “substantially” refers to the complete or nearly complete extent or degree of an action, characteristic, property, state, structure, item, or result. For example, in one embodiment, an object that is “substantially” located within a housing would mean that the object is either completely within a housing or nearly completely within a housing. The exact allowable degree of deviation from absolute completeness may in some cases depend on the specific context. However, generally speaking, the nearness of completion will be so as to have the same overall result as if absolute and total completion were obtained. The use of “substantially” is also equally applicable when used in a negative connotation to refer to the complete or near complete lack of an action, characteristic, property, state, structure, item, or result.

As used herein, the terms “approximately” and “about” generally refer to a deviance of within 5% of the indicated number or range of numbers. In one embodiment, the term “approximately” and “about”, may refer to a deviance of between 1-10% from the indicated number or range of numbers.

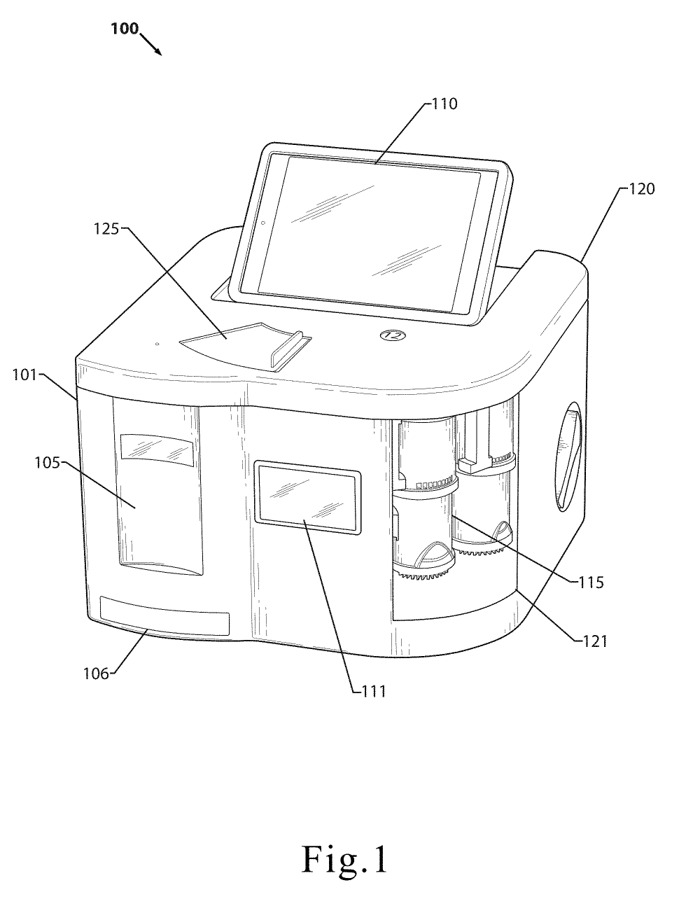

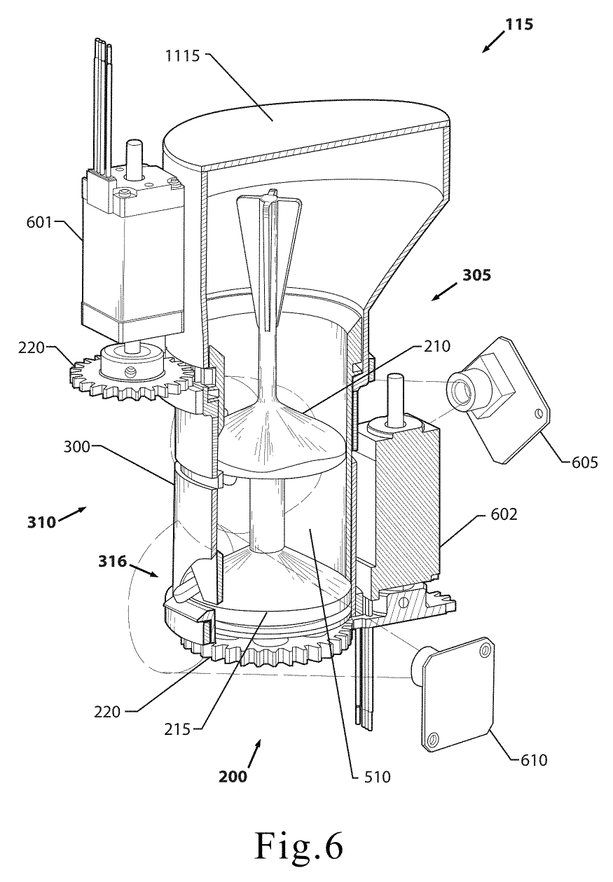

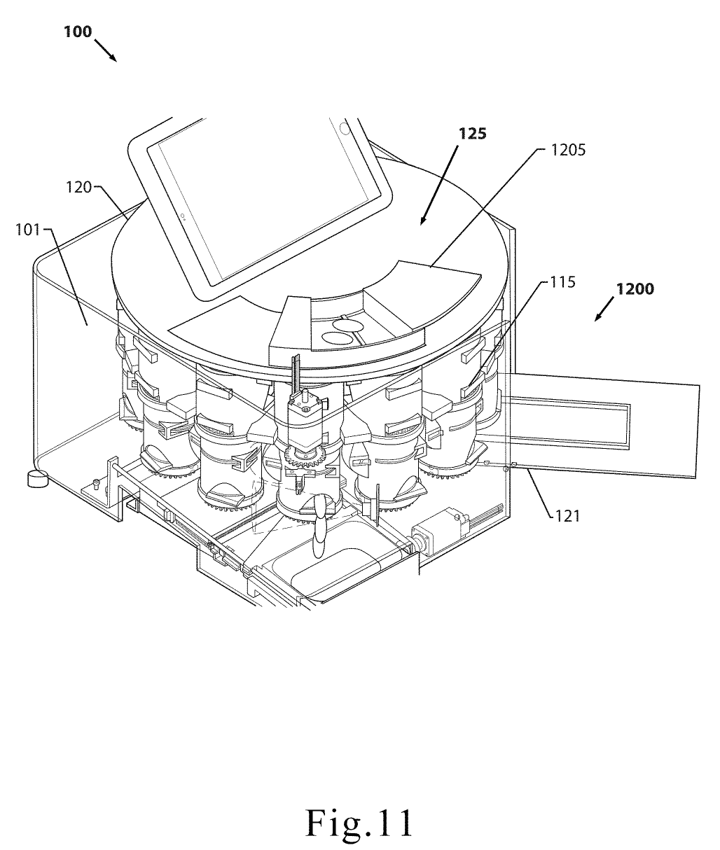

FIG. 1 is an illustration of a perspective view of one embodiment of the automated medication adherence system. As shown in FIG. 1, one embodiment of the automated medication adherence system 100 may comprise: a housing 101, a bar code reader 105, electronic interfaces 110, 111 a plurality of reservoirs 115, one or more sensors (shown in FIG. 6), and at least one pill delivery and lock-out module 106. The housing 101 may be any suitable shape and size for containing one or more reservoirs 115. For example, the housing 101 may be cubed shaped, as shown in FIG. 1. The housing 101 may define an exterior and an interior of the automated medication adherence system 100. The exterior may comprise a bottom surface that is capable of supporting the automated medication adherence system 100 on a flat surface, such as a desk or countertop. The exterior may comprise a top access cover 120, hereinafter referred to as an access cover, and a reservoir loading door 121 (shown in FIG. 11). The access cover 120 may be movable to an open position for manually placing reservoirs 115 into the interior or removing the reservoirs 115 or to maintain the system. The access cover 120 may preferably be movable to a closed and lockable position for preventing manual access to the reservoirs 115. Preferably, the reservoir loading door 121 may be used to load or remove reservoirs 115 by the end user. The lockable reservoir loading door 121 may be movable to an open position for manually placing or removing reservoirs 115 into the interior or removing the reservoirs 115 one at a time. Theft of prescription medicines by relatives of the prescription holder may be a problem solved by locking the access cover 120 and reservoir loading door 121. When the access cover 120 is in a closed lockable position, a pill loading assembly 125 located on at least one portion of the access cover 120 may provide access between the exterior of the housing 101 and the interior of the housing 101 in order to load medication into the reservoirs 115. Typically, gravity may be used to assist in loading medication through the pill loading assembly 125 into the reservoirs 115. The interior of the housing 101 may form an interior space that is sufficiently large to completely enclose the structural components of the automated medication adherence system 100. There may preferably be twelve reservoirs 115, which may preferably house at least sixty (60) of the largest prescribed pills each. A movable carrier, hereinafter referred to as a rotating carrier, may hold the reservoirs 115 in position wherein the electronic interfaces 110, 111 may actuate a motor to rotate the rotating carrier in order to align a pre-determined reservoir 115 with the pill loading assembly 125 of the access cover 120, such that medication may travel through the pill loading assembly 125 and into the correct, known, and identified reservoir 115. The rotating carrier may then rotate again to allow a user to load additional medications into different reservoirs 115. Each reservoir 115 may preferably house a homogenous type of medication, but this single type of medication may be one of many different sizes or shapes. The reservoirs 115 may comprise a medication preservation system to avoid cross-contamination, such as ultraviolet light protection, dust, excessive humidity, lids, and/or removable films. Additionally, in order to prevent contamination, a new reservoir 115 may be used each and every time a new medication is loaded and the medication may not contact or reuse any conduit except the pill loading assembly 125 of the access cover 120 and the pill delivery and lock-out module 106.

The reservoirs 115 are preferably configured to isolate a dosage of the medication contained in the specific reservoir 115 and then automatically deliver the dosage to the pill delivery and lock-out module 106. The dosage may be one pill or more than one pill, depending on the prescription. The system may repeat the dispensing process if multiple pills of the same type are simultaneously required. Preferably the rotating carrier rotates, such that the appropriate reservoir 115 is next to the pill delivery and lock-out module 106. The reservoir 115 may then deliver the dosage to the pill delivery and lock-out module 106. The rotating carrier may then rotate again, such that the next reservoir 115 may deliver a dosage of a different pill type. Once all of the dosages for that dosage time period have been delivered, the user may then take the dosage from the pill delivery and lock-out module 106. Under certain circumstances, it may be recommendable to deliver one pill type at a time, instead of all pills scheduled at the same time. The system algorithms may be capable to handle these instructions for use.

The housing 101 of the automated medication adherence system 100 may have a pill delivery and lock-out module 106, which may be a drawer, door, swing door, chute, and/or tray. The pill delivery and lock-out module 106 may be open or may be a locked portion, which is only unlocked for the specific user at a specific time. Once the entire dosage is in the pill delivery and lock-out module 106, the automated medication adherence system 100 may preferably notify the user to retrieve the dosage. This notification may be an audible alert, visual alert, vibration, and/or a wireless electronic communication to an electronic device used by the user. The pill delivery and lock-out module 106 may have one or more sensors that determine the status of the dosages within the pill delivery and lock-out module 106. The pill delivery and lock-out module 106 opens to deliver medications to user.

The pill delivery and lock-out module 106 may comprise a lock-out tray that opens in the event that a dosage is not removed from the dispensing tray configuration of the pill delivery and lock-out module 106 by the user. In this manner, the next dosage does not get mixed up with the missed dosage, avoiding an overdose. The system may record all medication events and a high frequency of pills transferred to the lock-out module may constitute a pattern of non-adherence. Preferably, if medication non-adherence becomes an issue, a notification may be sent to the user, pharmacy, care giver and/or a health care provider. In this manner, non-adherence can be dealt with appropriately.

Although a rotating carrier is shown as the mechanism that moves the reservoirs 115 within housing 101, the motion of the reservoirs 115 may be accomplished by other devices, including actuators, pulleys, slides, and the like.

A bar code reader 105 may be positioned on the exterior of the housing 101, or at another appropriate location, to read the medication prescription record number and other bar-coded information needed for automatic programming and ease of use by a user of the automated medication adherence system 100. For example, the bar code reader 105 may enable the recognition of data relating to the medication, including a pill identity, pill type, pill size, pill shape, a user identity, a dosage schedule, dosage information, and potential side effects that may be used to automatically program the automated medication adherence system. Therefore, the automated medication adherence system 100 does not require any programming by a user. Prior to loading medication into a reservoir 115, the bar code reader 105 may allow the user to send the data relating to the medication to the electronic interface 110 for programming each of the reservoirs 115 with the specific information required to accurately dispense the medication to be loaded.

Likewise, the electronic interfaces 110, 111 may be positioned on the exterior of the housing 101, or at another appropriate location, for ease of use by a user of the automated medication adherence system 100. FIG. 1 shows that the electronic interfaces may be a permanent fixture and/or a removable hand-held computing device. The electronic interfaces 110,111 may be used for accomplishing various interface and notification functions. For example, the electronic interface 110,111 may also be manually programmed with data relating to the medication, including a pill identity, pill type, pill size, pill shape, pill images, schedule time, daily frequency, a user identity, a dosage schedule, dosage information, not to exceed amounts, instructions for use and potential side effects. The electronic interfaces 110,111 may enable programming of each of the reservoirs 115 with the specific information about the medication to be held in the respective reservoir 115. The electronic interface may facilitate the openings, agitation, and rotational parameters of each reservoir 115 to match the geometry, size and construction of each pill type so each reservoir 115 may be capable of accurately dispensing any pill type. The electronic interfaces 110, 111 may comprise a computing component and a display/interactive component. The computing component may control the one or more reservoirs 115, such that the precise dosage of medication is delivered from the reservoirs 115 to the pill delivery and lock-out module 106 each and every time on the dosage schedule. The display/interactive components may preferably be a touch screen so the patient can acknowledge and authorize certain steps, initiate certain actions, and provide a high level of interactivity and operability.

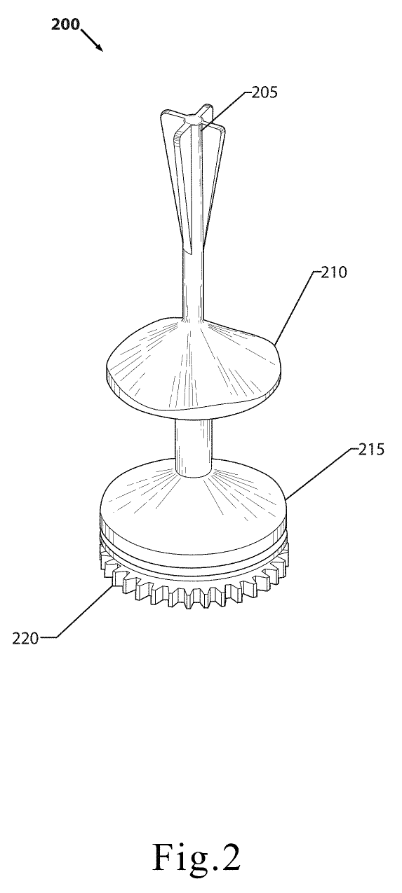

FIG. 2 is an illustration of a perspective view of one embodiment of a central agitation stalk for the reservoirs. FIG. 2 shows that each reservoir within the automated medication adherence system may comprise a central agitation stalk 200. The central agitation stalk 200 may generally provide rotation and agitation within the reservoir, such that each reservoir may receive, store, and dispense pills, tablets, and capsules of various sizes and geometries accurately and precisely. The central agitation stalk 200 may comprise fin portion 205, a wave surface 210, a ribbed cone surface 215, and gear teeth 220. When a user loads medication into a reservoir, the fin portion 205 may prevent the medication from inadvertently clumping together. The medication may generally fall onto the wave surface 210 and be stored until the electronic interface causes the reservoir to dispense the medication. At a desired and/or scheduled time period, a drive mechanism engages with gear teeth 220 and causes the central agitation stalk 200 to rotate in order to move the medication on the wave surface 210. This rotational movement may be combined with an agitation movement in order to move—one pill at a time—the medication from the first stage (wave surface 210) to the lower or second stage (ribbed cone surface 215). The medication may reside on the ribbed cone surface 215 until it is ready to be dispensed, one pill at a time, into a pill delivery and lock-out module. The fin portion 205, wave surface 210, and ribbed cone surface 215 may be rotated and/or agitated simultaneously, or separately, such that only one or two rotate, while the others remain still.

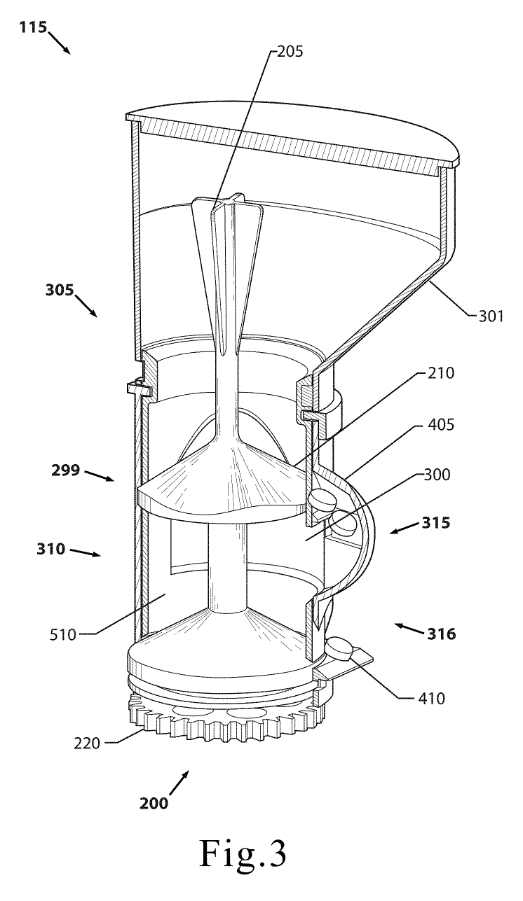

FIG. 3 is an illustration of a cross-section view of one embodiment of a reservoir that is contained within the automated medication adherence system. FIG. 3 shows that each reservoir 115 within the automated medication adherence system may comprise a generally cylindrical two-stage device for storing and dispensing medication. The cylindrical reservoir 115 may comprise a stalk housing 299, also referred to as a reservoir housing, and a central agitation stalk 200. As shown in FIG. 3, the stalk housing 299 may comprise a funnel shaped hopper 301 and two overlapping wall cylinders, outer wall cylinder 300 and inner wall cylinder 510. The central agitation stalk 200, inner wall cylinder 510, and outer wall cylinder 300 may delineate the boundaries of the first stage 305 and the second stage 310. The first stage 305 may comprise a receptacle for storing medication, and then transfer the medication, in a controlled manner, to the second stage 310. Once the reservoir 115 to be loaded with medication is aligned with the pill loading assembly in the access cover, a user may load medication via the top of the reservoir 115 into the first stage 305 of the reservoir 115. FIG. 3 shows that the hopper 301 may be wide and funnel shaped in order to allow loading of the medication through the access cover without spilling any pills or medication and to maximize storage capacity. The medication may fall past the fin portion 205 and the central agitation stalk 200 may rotate in order for the medication to settle onto the wave surface 210. The first stage 305 may also comprise a first through-hole 315 around the periphery such that the medication may exit the first stage 305 and enter the second stage 310. Sensors, rotational and agitation actuators, and logic algorithms may ensure that only a specified number of pills, usually one or two pills, are transferred—one pill at a time—from the first stage 305 to the second stage 310. The second stage 310 may comprise a second through-hole 316 for allowing the medication to be dispensed, one pill at a time, to the pill delivery and lock-out module. The first and second through-holes 315, 316 may be created when openings in the inner and outer wall cylinders 510, 300 overlap through rotational motion to create a through-hole that best matches the pill geometry. Proprietary algorithms may use information from the pharmacy prescription records and pharmaceutical and/or proprietary databases to calculate the reservoir 115 through-holes 315, 316 and certain agitation and rotation parameters required to dispense the pills with high degree of accuracy. FIG. 3 also shows that the through-holes 315, 316 may comprise or be connected to chutes 405 and 410. As the medication pill passes through through-holes 315 or 316, the chutes 405, 410 may direct it to travel along a predetermined path.

In other embodiments, the stalk 200 housing may be of a unitary design, wherein rotational, actuation, and agitation are used to move the medication from the first stage 305 to the second stage 310 and from the second stage 310 to the pill delivery and lock-out module.

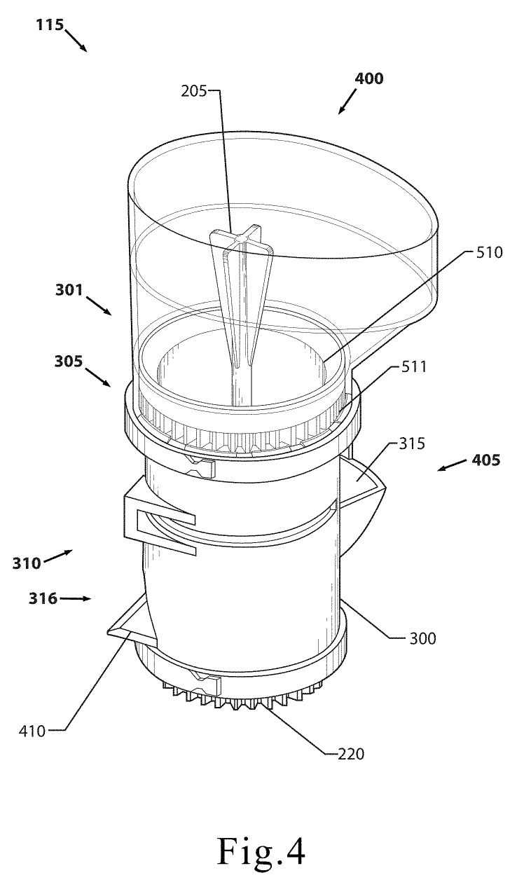

FIG. 4 is an illustration of a perspective view of one embodiment of a reservoir that is contained within the automated medication adherence system. FIG. 4 shows that each reservoir 115 within the automated medication adherence system may comprise a hopper 301 and a generally cylindrical outer wall cylinder 300. The hopper 301 may comprise or be connected to a top portion 400 of the reservoir 115. The top portion 400 may be covered by a rotating reservoir cover assembly 1105 that simultaneously opens and closes all reservoirs 115, allowing access from the pill loading assembly to the reservoir 115 when the rotating reservoir cover assembly 401 is open and preventing contamination and allowing transportation when the rotating reservoir cover assembly 1105 is closed. Additionally, the outer wall cylinder 300 may comprise or otherwise be connected to one or more chutes 405, 410. The first stage 305 of the reservoir 115 may comprise a first chute 405 to help guide medication traveling from the first stage 305 to the second stage 310. The first chute 405 may be an extension of the first through-hole 315. Likewise, the second stage 310 of the reservoir 115 may comprise a second chute 410 to help guide medication being dispensed into a pill delivery and lock-out module. The second chute 410 may be an extension of the second through-hole 316.

FIG. 4 also shows show the inner wall cylinder 510 may be comprised of gear teeth 511 and how the inner wall cylinder 510 may be substantially contained within the outer wall cylinder 300, such that the inner wall cylinder 510 may be turned, via gear teeth 511, within the outer wall cylinder 300. As shown, the gear teeth 511 may be preferably accessible through the outer wall cylinder 300. FIG. 4 also shows how the gear teeth 220 may be accessible through the outer wall cylinder 300.

FIG. 4 also shows how the hopper 301 may be designed to contain and be filled with medication, which may be prevented from clumping by the rotation of the fin portion 205. The agitator

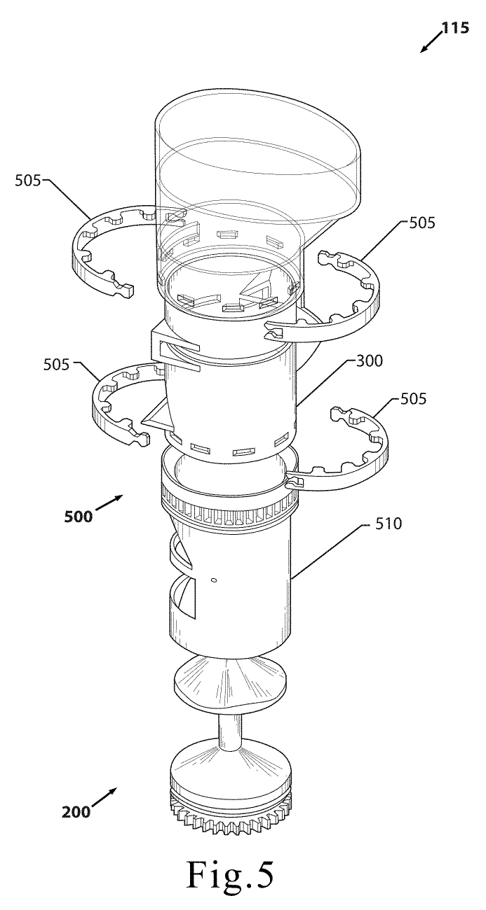

FIG. 5 is an illustration of an exploded view of one embodiment of a reservoir that is contained within the automated medication adherence system. FIG. 5 shows that each reservoir 115 within the automated medication adherence system may comprise a multi-component receptacle. FIG. 5 shows that the reservoir 115 may comprise a central agitation stalk 200, wall cylinders 300, 510, and one or more retaining rings 505. The central agitation stalk 200 may generally provide a rotational and agitation motion such that each reservoir 115 may receive, store, and dispense medication in an extremely precise and accurate manner. The outer wall cylinder 300 and the inner wall cylinder 510 may rotate relative to each other, which in turn, may create different sizes of through-holes around the periphery such that medication may pass between from the first stage to the second stage or be dispensed into the pill delivery and lock-out module. The retaining rings 505 may provide support to the top and bottom portions of the outer wall cylinder 300 and assist the outer wall cylinder in containing inner wall cylinder 510. The rings 505 may also hold the stalk vertically in place within the inner wall cylinder 510, as shown in FIG. 6.

FIG. 6 is an illustration of a cross-section view of one embodiment of the interior of the automated medication adherence system. FIG. 6 shows that the automated medication adherence system may comprise one or more sensors 605, 610 and one or more actuators 601, 602. The sensor 605 may be configured to sense when a single pill, or a specific dosage of pills moves from the first stage 305 to the second stage 310. The sensor 605 may be an optical sensor, preferably a camera sensor, but other types of sensors may be used. The sensor 605 may preferably be positioned to monitor movement and ejection of a pill through the first through-hole 315. The sensor 610 may be configured to sense when a single pill, or a specific dosage of pills moves from the second stage 310 to the pill delivery and lock-out module. The sensor 610 may be an optical sensor, preferably a camera sensor, but other types of sensors may be used. The sensor 610 may preferably be positioned to monitor movement and ejection of a pill through the second through-hole 316.

The sensors 605, 610 are preferably connected to the computing component of electronic interface, such that the automated medication adherence system can detect when a pill has transferred to the second stage 310 or to the pill delivery and lock-out module.

FIG. 6 also shows how the actuators 601 and 602 interconnect with the inner wall cylinder 510 and the central agitation stalk 200, respectively, through the gear teeth 511 and 220, respectively. The actuators 601, 602 are controlled by the computing component of the electronic interface, such that the automated medication adherence system may accurately and precisely dispense medication of almost any size or shape. The actuator 601, as shown, may cause the inner wall cylinder 510 to rotate, such that the first and/or second through-hole 316 may be created and/or closed as needed to move the medication and control the ejection through the automated medication adherence system. The outer wall cylinder 300 and the inner wall cylinder 510 each may have openings that, when aligned by the rotation of the inner wall cylinder 510, create through-holes 316. The actuator 601 may also provide agitation, which may de-clump the medication, in the event that a sensor 605, 610 detects such clumping, and/or that may cause the medication to eject through the through-holes 315, 316 in a controlled manner.

The actuator 601, as shown, may cause the wave surface 210 to rotate, such that the medication resting on the wave surface 210 is brought to the first through-hole. Though agitation and rotation (backward and/or forward) of actuators 601 and/or 602, the pill on the wave surface 210 may be caused to go through the first through-hole and down to the second stage 310, ideally one pill at a time. The sensor 605 may then inform the computing component that a single pill has successfully been moved and the actuator 601 may then close the first through-hole by reversing (or continuing) the rotation of the inner wall cylinder 510. Similarly, the actuator 602, as shown, may cause the ribbed cone surface 215 to rotate, such that the medication resting on the ribbed cone surface 215 may be brought to the second through-hole 316. Though agitation and rotation of the actuators 602 and/or 601, only one pill on the ribbed cone surface 215 may be caused to go through the second through-hole 316 and out to the pill delivery and lock-out module. The sensor 610 may then inform the computing component that the single pill has successfully been moved and the actuator 602 may then close the second through-hole 316 by reversing (or continuing) the rotation of the inner wall cylinder 510. This process may be repeated until the correct dosage has been delivered from to the pill delivery and lock-out module.

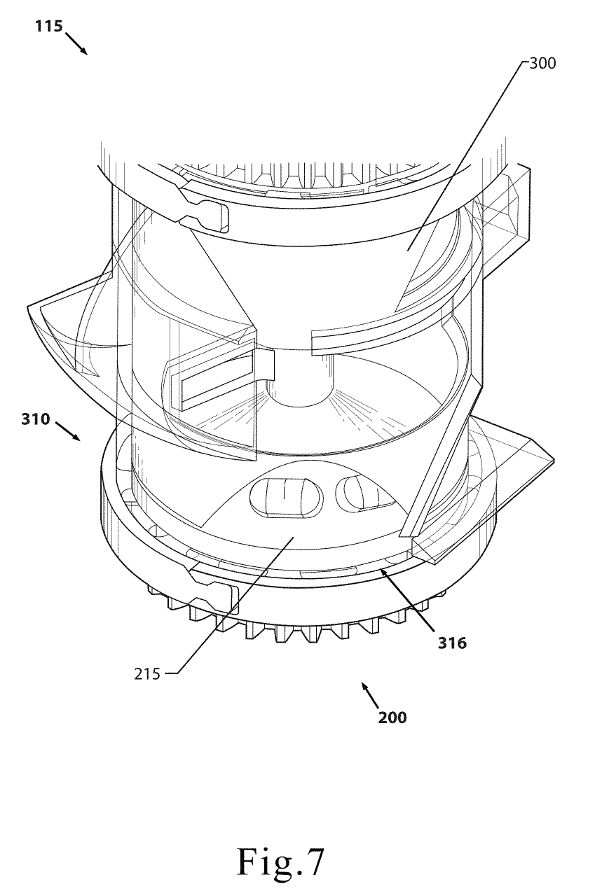

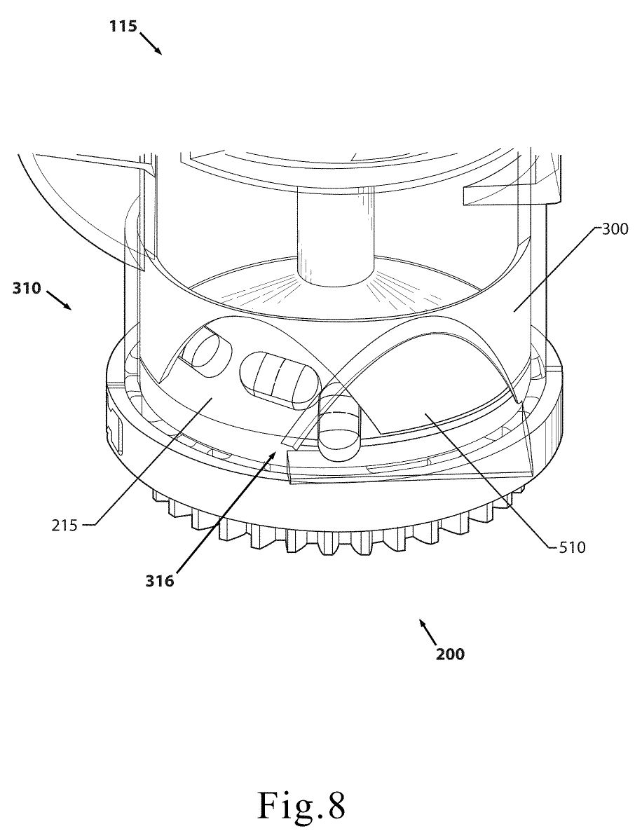

FIG. 7 is an illustration of a close-up view of one embodiment of the second stage of a reservoir that is contained within the automated medication adherence system. FIG. 7 shows that the second stage 310 of a reservoir 115 may house medication on the ribbed cone surface 215 of the central agitation stalk 200 prior to dispensing the medication into the pill delivery and lock-out module. Preferably, the amount of medication on the ribbed cone surface 215 is only a small number of pills and may be a single dosage of the medication to be delivered to the pill delivery and lock-out module. The actuator may align the outer wall cylinder 300 and the inner wall cylinder of the reservoir 115 such that the through-hole 316 (shown in FIG. 8) is not yet formed. Thus, the medication in the second stage 310 cannot yet exit. The ribbed cone surface 215 may be agitated by the actuator at specific amplitudes and frequencies in order to facilitate the separation of the medication on the ribbed cone surface 215, in order to line up one pill behind the other so that the medication can be transferred, one pill at a time, to the pill delivery and lock-out module. The actuator may continue (or reverse) the rotation of the ribbed cone surface 215 until each pill conforms to its exit position, preferably lengthwise. Additionally, using pre-programmed medication data and algorithms, the rotation and agitation parameters may adjust the through-hole 316 to best match the dimensions of the pill.

FIG. 8 is an illustration of a close-up view of one embodiment of a reservoir that is contained within the automated medication adherence system with continuously variable through-holes to best match the solid geometry of the pill loaded into a specific reservoir. FIG. 8 shows that the second stage 310 of a reservoir 115 may house medication on the ribbed cone surface 215 of the central agitation stalk 200 prior to dispensing the medication into a pill delivery and lock-out module. The outer wall cylinder 300 and the inner wall cylinder 510 of the reservoir 115 may be aligned such that the second through-hole 316 around the periphery is formed and accessible for medication dispensing at a desired period of time. When the medication reaches the second through-hole 316 around the periphery, the medication, through gravity, may pass through the second through-hole 316. Agitation may be provided by the central agitation stalk 200 in order to assist the medication in passing through the second through-hole 316. Algorithms for agitation and rotation parameters may be pre-programmed into the electronic interface and may include specific amplitudes and frequencies in order to facilitate the travel of the medication through the automated medication adherence system. The automated medication adherence system may accommodate medication generally ranging from about 3 to about 28 millimeters, but the reservoir size, through-holes, and agitation and rotation parameters may be changed to increase the range of pill sizes and solid geometries. Once the dosage of medication has been dispensed, the electronic interface may close the second through-hole 316 and provide an alert that the medication is available for consumption.

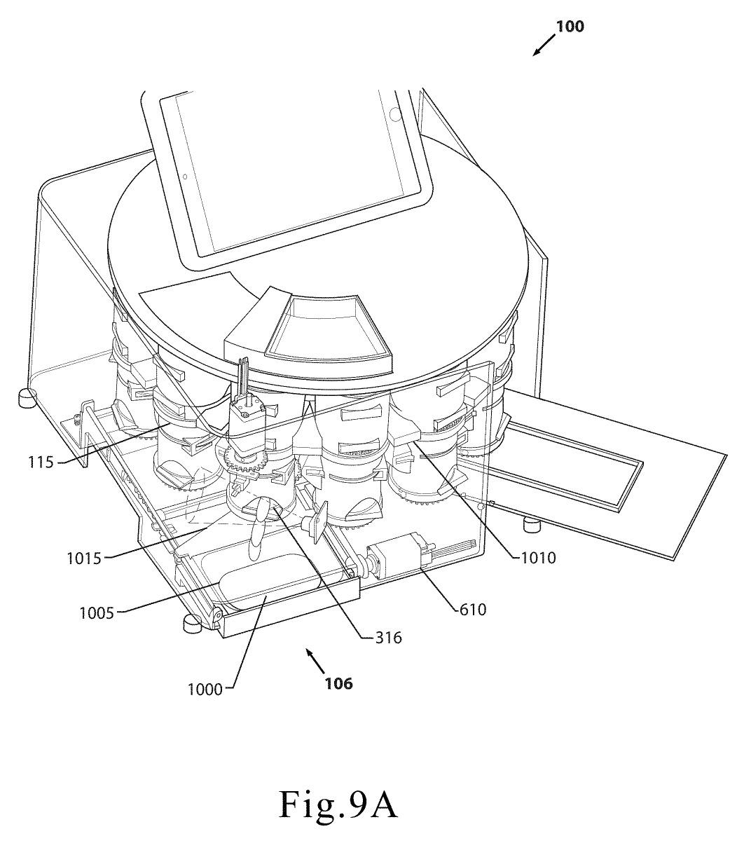

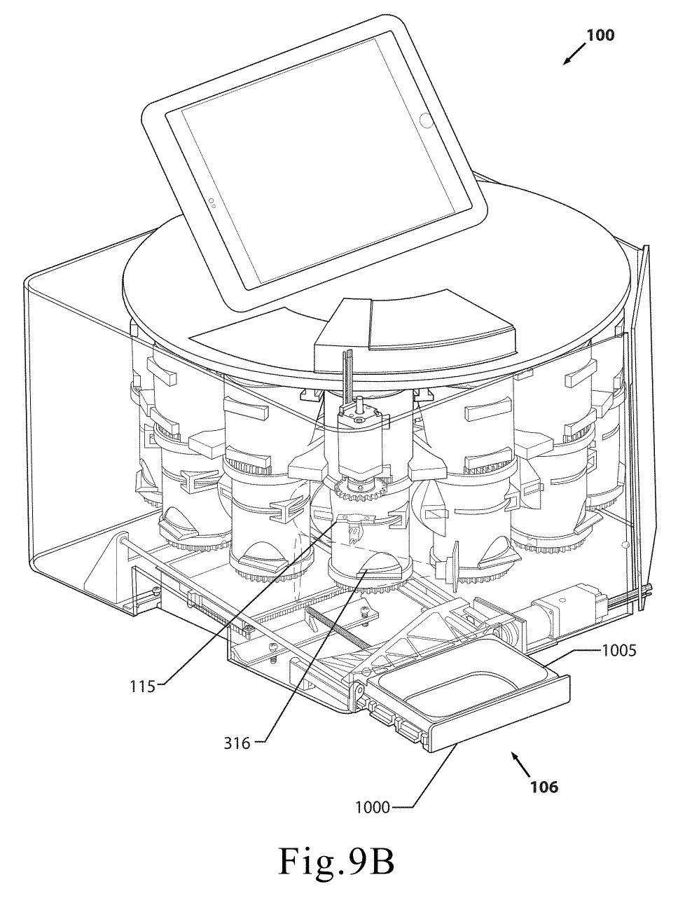

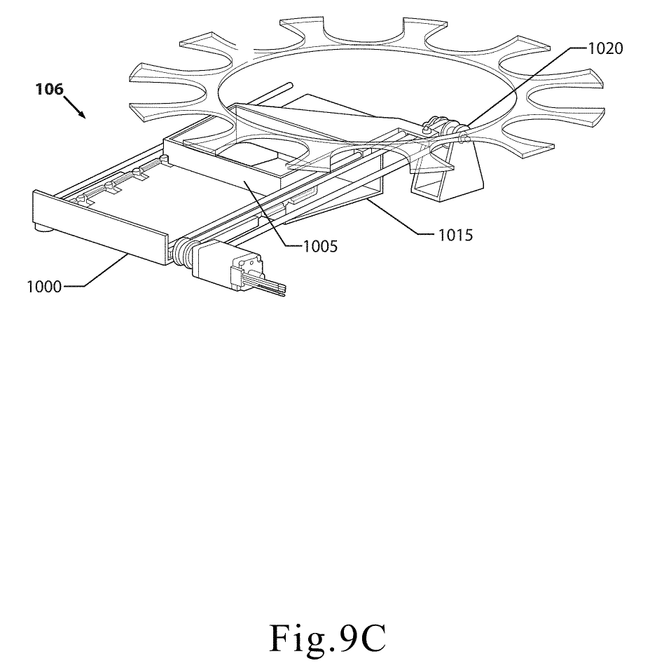

FIG. 9A is an illustration of a close-up view of one embodiment of the pill delivery and lock-out module in its neutral (holding pill delivery and lock-out module) position that is contained within the automated medication adherence system. FIG. 9A shows that the reservoirs 115 are preferably configured to isolate a dosage of medication contained in the specific reservoir 115 and then deliver the dosage to the pill delivery and lock-out module 106. The pill delivery and lock-out module 106 may comprise a dispensing tray 1000, a transporter 1005, and a lock-out tray, which is created by the axial movement of the transporter 1005 in relation to the other components described below. The pill delivery and lock-out module 106 may be substantially housed within the interior of the automated medication adherence system 100 when holding medication. Preferably, the rotating carrier 1010 rotates, such that the appropriate reservoir 115 may be next to the pill delivery and lock-out module 106. Then an actuator may cause the reservoir 115 to rotate, such that the medication resting on the ribbed cone surface may brought to the second through-hole 316. Through agitation and rotation of the actuators, the medication on the ribbed cone surface may be caused to go through the second through-hole 316 and out to the pill delivery and lock-out module 106. The dosage of medication may be one or more pills. The dosage may come from one or more reservoirs 115. Once the entire dosage is within the dispensing tray 1000, the user may then be signaled to collect the dosage. A sensor 610 may inform the computing component that the medication has successfully been moved and the actuator may close the second through-hole 316 by rotation of the inner wall cylinder of the reservoir 115. This process may be repeated until the correct dosage of medication(s) has been delivered to the pill delivery and lock-out module 106. The sensor 610 may preferably be positioned to monitor movement of medication through the second through-hole 316 to the pill delivery and lock-out module 106. The sensor 610 may take pictures and store data confirming that a dosage and/or total dosage of medication was dispensed. When the prescribed dose of medication for that dosage time period has been delivered to the pill delivery and lock-out module 106, the pill delivery and lock-out module 106 may move to the exterior of the automated medication adherence system 100 to a forward position so a user may then take the dosage of medication from the pill delivery and lock-out module 106. Additionally, notification may alert a user the medication is ready for consumption.

FIG. 9A also shows that if one or more pills were erroneously dispensed, the pill delivery and lock-out module 106 may remain in the neutral position, below the reservoir 115, and hold the pills that were dispensed from the second through-hole 316. The transporter 1005 may also move in reverse to transfer any erroneously dispensed pills to a lock-out tray 1015 (shown in FIG. 9C). Preferably, when the lock-out tray 1015 operates, a notification may be sent to the user, any authorized individual, and/or a health care provider. In this manner, missed or incorrect dosages may be dealt with appropriately.

FIG. 9B is an illustration of a close-up view of one embodiment of the pill delivery and lock-out module in a forward position. FIG. 9B shows when one or more pills are dispensed from the second through-hole 316 of a reservoir 115, the transporter 1005 may move forward to eject the dispensing tray 1000 into a forward position when the patient is ready to take the pills. If the pill delivery and lock-out module 106 is in the forward position and the pills are not removed by the patient after a period of time, the transporter 1005 may also move in reverse to transfer the pills to a lock-out tray 1015. A dosage of medication may rest within the pill delivery and lock-out module 106 until the medication is ready to be collected by a user.

FIG. 9C is an illustration of one embodiment of the pill delivery and lock-out module in its reverse (lock-out tray) position that is contained within the automated medication adherence system. FIG. 9C shows that if a dosage of medication is not retrieved after a pre-determined period of time or if the dosage of medication has been dispensed incorrectly, the transporter 1005 may move or slide backward and guide the medication from the dispensing tray 1000 of the pill delivery and lock-out module 106 to a lock-out tray 1015. Once the medication is stored in the lock-out tray 1015, a user adherence record may be updated and the transporter 1005 may move back into its neutral position. In this manner, the next dosage may not get mixed up with the previous dosage, avoiding an over dosage, and an incorrect dosage may be held in the lock-out tray 1015. Preferably, when the lock-out tray 1015 operates, a notification may be sent to the user, one or more authorized individuals, and/or one or more health care providers. In this manner, missed or incorrect dosages may be dealt with safely and appropriately and double or wrong dosage is prevented. FIG. 9C shows that actuator 1020 may be used to slide the transporter 1005 back and forth to dispense or retrieve an unused or incorrect dosage.

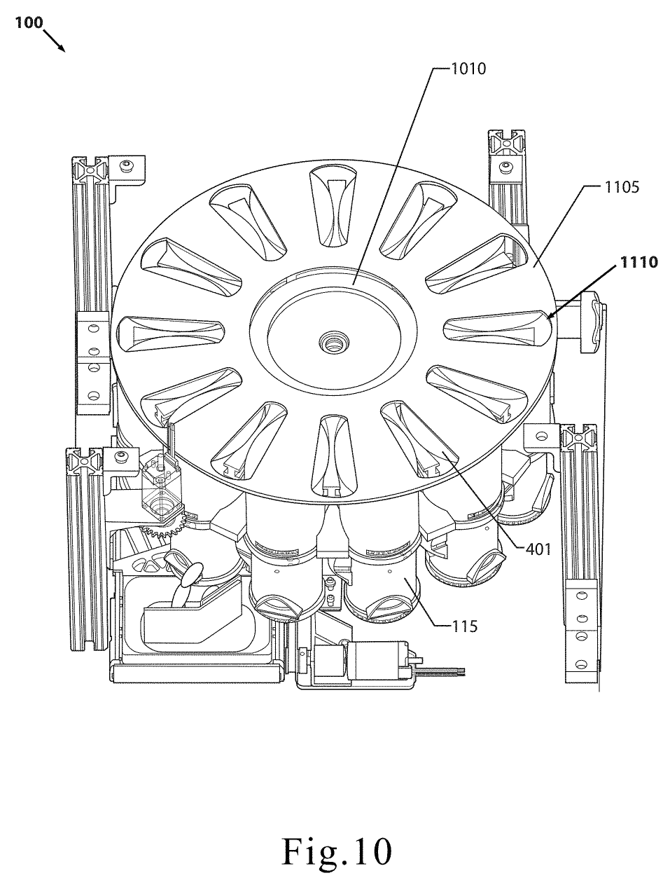

FIG. 10 is an illustration of one embodiment of the reservoir cover mechanism that opens and closes a reservoir fill opening that is contained within the automated medication adherence system. FIG. 10 shows the automated medication adherence system 100 may comprise an access cover. The access cover may be movable to an open or closed position. When the access cover is in a closed lockable position, a pill loading assembly located on at least one portion of the top access cover may provide access between the exterior of the housing and the interior of the housing in order to load medication into the reservoirs 115. A rotating carrier 1010, may hold the reservoirs 115 in position wherein the electronic interface may actuate a motor to rotate the rotating carrier 1010 in order to align a pre-determined reservoir 115 with the pill loading assembly of the access cover, such that medication may travel through the pill loading assembly and into the correct, known, and identified reservoir 115. The rotating carrier 1010 may then rotate again to allow a user to load additional medications into different reservoirs 115. The interior of the housing may also comprise a rotary lid 1105. The rotary lid 1105 may be positioned above the reservoirs 115 and may passively rotate in response to the clockwise or counter clockwise rotation of the rotating carrier 1010. The rotary lid 1105 may provide access to the interior of the reservoirs 115 when the openings 1110 in the rotary lid 1105 align with the openings of the rotating reservoir cover assembly 401. Likewise, the rotary lid 1105 may prevent access to the interior of the reservoirs 115 when the openings 1110 in the rotary lid 1105 do not align with the openings of the rotating reservoir cover assembly 401. An additional fixed cover may be present above the rotary lid 1105.

FIG. 11 is an illustration of one embodiment of the automated medication adherence system showing the reservoir loading door and the pill loading assembly. FIG. 11 shows the reservoir loading door 121 may be used to load or remove reservoirs 115 by the end user. The reservoir loading door 121 may be movable to an open position 1200 for manually placing or removing reservoirs 115 into the interior of the automated medication adherence system 100 or removing the reservoirs 115 one at a time. Preferably, the reservoir loading door 121 may slide individual reservoirs 115 in and/or out of the interior of the automated medication adherence system 100. When the reservoir loading door 121 is not in use, the reservoir loading door 121 may fold up in a vertical direction and form part of the exterior of the housing 101.

FIG. 11 also shows when the access cover 120 is in a closed lockable position, a pill loading assembly 125 located on at least one portion of the access cover 120 may provide access between the exterior of the housing 101 and the interior of the housing 101 in order to load medication into the reservoirs 115. Typically, gravity may be used to assist in loading medication through the pill loading assembly 125 into the reservoirs 115. The pill loading assembly 125 may also comprise a pill wiper 1205, which may wipe medication into the reservoirs 115. When the pill loading assembly 125 is not in use, the pill wiper 1205 may remain in a closed position, preventing access to the pill loading assembly 125.

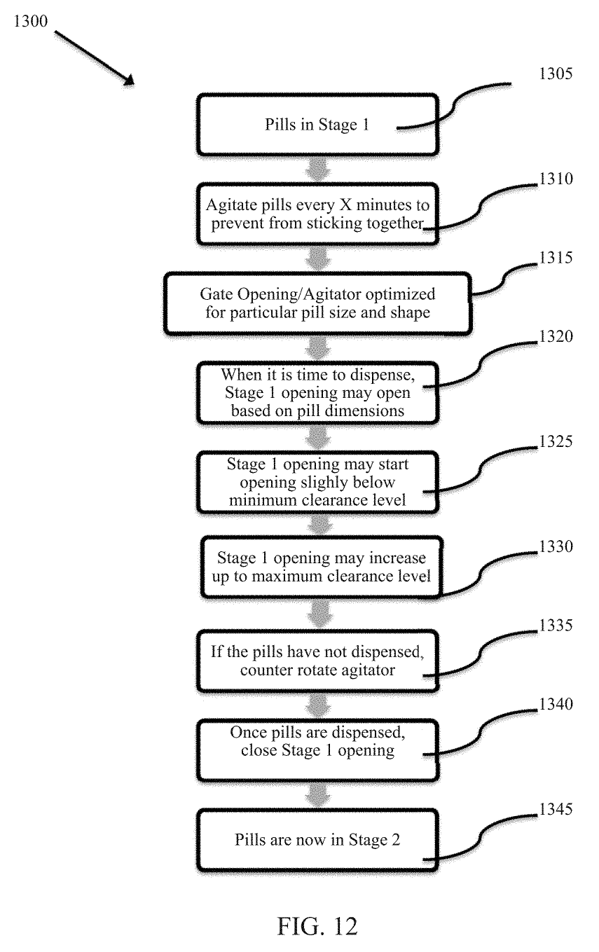

FIG. 12 is a flow block diagram of one embodiment of the method of medication moving through the first stage of the automated medication adherence system 1300. FIG. 12 shows that after a user loads medication into the automated medication adherence system, the medication may remain in the first stage 1305 of the reservoir for storage until the medication is ready to be transferred to the second stage. The agitator may agitate the reservoir using proprietary algorithms at set time intervals in order to prevent the medication from sticking to one another 1310. Similarly, the reservoir may agitate using calculated algorithms to begin transporting the medication toward the through-hole in the first stage using calculated rotation and counter rotation parameters. Preferably, the agitator may be optimized for a particular pill size and shape 1315. Accordingly, the through-hole in the first stage may start opening based on the medication's dimensions 1320. The through-hole may increase from a minimum clearance level 1325 until it has reached the maximum clearance level 1330. If the medication has not dispensed into the second stage, the agitator may counter rotate a number of full turns 1335. This may help the pills realign on the surface and may clear pill jams within the first stage. The sensor may detect when a single pill has been dispensed and immediately close the through-hole in the first stage. The medication should now be in stage two 1345.

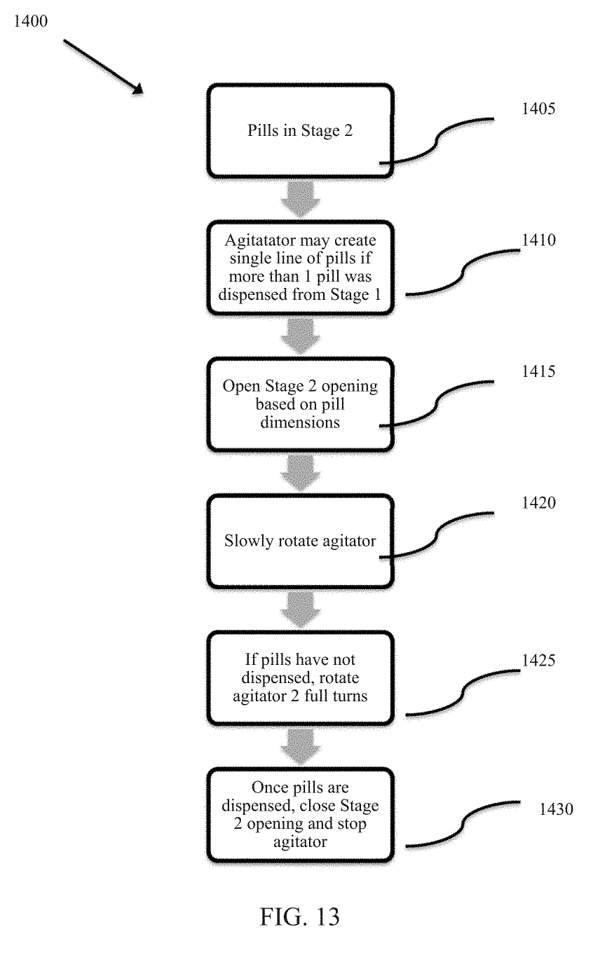

FIG. 13 is a flow block diagram of one embodiment of the method of medication moving through the second stage of the automated medication adherence system 1400. FIG. 13 shows that after that the medication may travel from the first stage to the second stage 1405. Once the medication is in the second stage, the agitator may rotate in order to line the medication up in single line along the surface of the second stage, if more than one pill was transferred from the first stage to the second stage 1410. The through-hole of the second stage may open based on the medication's dimensions 1415 and the agitator may slowly begin to rotate 1420 in order to help the medication dispense into the pill delivery and lock-out module. If the medication did not dispense, the agitator may rotate for two full turns 1425 in order to dispense the medication. A sensor may detect when a single pill has been dispensed into the pill delivery and lock-out module and the through-hole of the second stage may close and the agitator may stop rotating 1430. The pills may fall out via gravity from the second stage through-hole as the pills are rotated past the second stage through-hole.

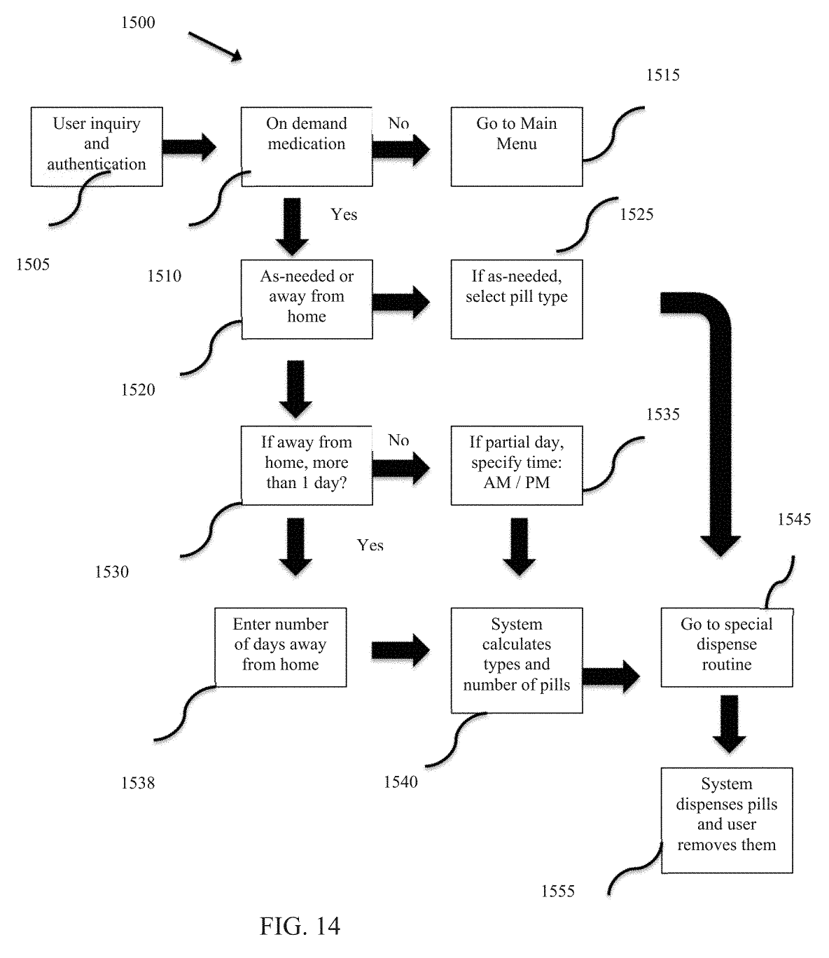

FIG. 14 is a flow block diagram of one embodiment of the method of taking medication on an as-needed or away from home basis 1500. When a user engages with the automated medication adherence system, the automated medication adherence system may inquire as to the user's identity and authentication 1505. Although the automated medication adherence system does not require the user to do any programming, the user may be required to input, scan, or otherwise upload information relating to the user, the medication, the prescribing entity, and/or the prescription. If a user would like the use the automated medication adherence system for on demand medication 1510, the user may be directed to the Main Menu of the computing component 1515. Otherwise, the user may select a medication event such as an as-needed basis or away from home basis 1520. If the user decides to take the medication on an as-needed basis, the user may be prompted to select a pill type 1525. The medication may then undergo a special dispensing routine 1545. The automated medication adherence system may then dispense the medication and a user may remove the medication for consumption 1555. If the user has exceeded the maximum number of on-demand medications allowed over a pre-determined period of time, the system may not dispense anymore medication and may alert the user and a medical professional of this condition. If the user decides to take the medication on an away from home basis, the user may have to decide whether they will be away from home for more than one day 1530. If the user may be away from home for more than one day, the user may enter the number of days they will be away from home 1538. If the user will not be away from home for more than one day, the user may specify when they will be away, for example, in the morning and/or the evening 1535. The automated medication adherence system may then calculate the type and number of pills required by the user 1540. The medication may then undergo a special dispensing routine 1545. The automated medication adherence system may then dispense the medication and a user may remove the medication for consumption 1555.

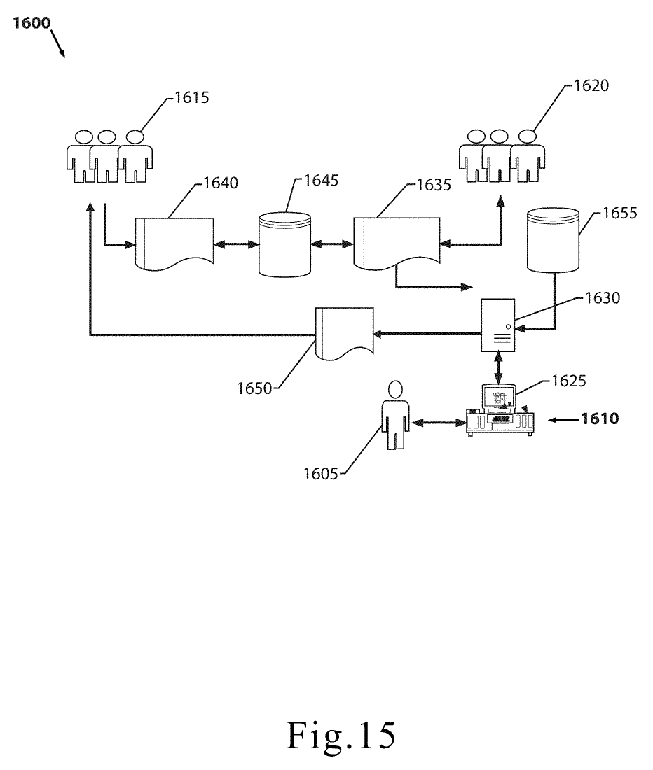

FIG. 15 is a flow block diagram of one embodiment of the method of programming the reservoirs and dispense logic using one or more algorithms using the computing component 1600. FIG. 15 shows the user information and data relating to the medication may be transmitted to and from an automated medication adherence system 1610, by one or more health care providers 1615, and/or pharmacists 1620 through an online connection, bar code scan, or direct upload. For example, the electronic interface 1625 may be manually programmed with data relating to the medication, including a pill identity, pill type, pill size, pill shape, pill images, schedule time, daily frequency, a user identity, a dosage schedule, dosage information, not to exceed amounts, instructions for use and potential side effects. The electronic interface 1625 may enable programming of each of the reservoirs with the specific information about the medication to be held in the respective reservoir. Additionally, the electronic interface may utilize a bar code reader, positioned on the exterior of the automated medication adherence system 1610, or at another appropriate location, to read the medication prescription record number and other bar-coded information needed for automatic programming and ease of use by a user 1605 of the automated medication adherence system 1610. For example, the bar code reader may enable the recognition of data relating to the medication, including a pill identity, pill type, pill size, pill shape, a user identity, a dosage schedule, dosage information, and potential side effects. Therefore, the automated medication adherence system 1610 does not require any programming by a user 1605. Prior to loading medication into a reservoir, the bar code reader may allow the user 1605 to send data relating to the medication to the electronic interface 1625 for programming each of the reservoirs with specific information about the medication to be loaded.

The data relating to the medication may be stored in a cloud application 1630. The cloud application 1630 may also receive data relating to the medication from a pharmacy software interface 1635. When a health care provider 1615 writes a prescription for the user 1605, the prescription may be stored in a health care provider software interface 1640 and transmitted as an e-prescription 1645 to the pharmacy software interface 1635. The pharmacy software interface 1635 may work in conjunction with a pharmacist 1620 to dispatch the appropriate medication for the user 1605. Additionally, the pharmacy software interface 1635 may transmit the e-prescription 1645 to the cloud application 1630 for programming the electronic interface 1625 of the automated medical adherence system 1610. Data relating to the medication may also be stored in a medication database 1655.

FIG. 15 also shows that the automated medication adherence system 1610 may be in communication with a health care provider 1615 to allow the health care provider 1615 to perform various modifications to the programming of the automated medication adherence system 1610 from a remote location. Additionally, this communication may alert a health care provider 1615 to problems, such as when the user 1605 fails to remove one or more dosages of medication from the pill delivery and lock-out module. Communication is preferably achieved by sending and receiving medication events 1650 via a cloud application 1630.

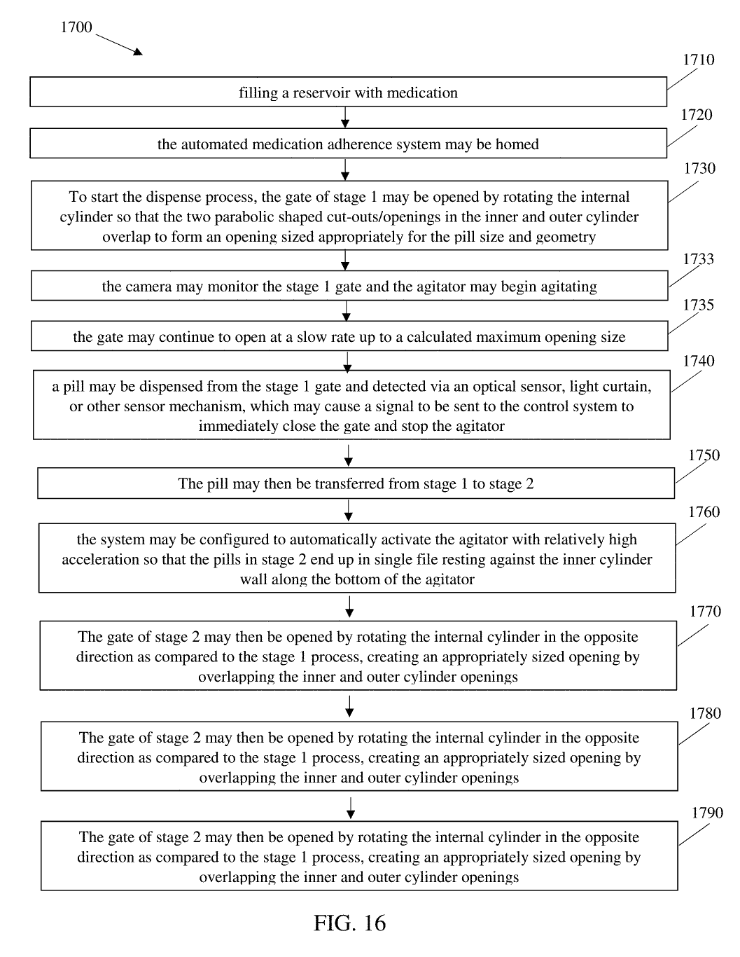

FIG. 16 is a flow block diagram of one embodiment of a method of dispensing medication using the automated medication adherence system 1700. As shown in FIG. 16, the method of dispensing medication using the automated medication adherence system 1700 may comprise the step of filling a reservoir with medication, which may be pills 1710. The pills 1710 may be tablets, caplets, coated or uncoated pills, gel caps, capsules, and the like.

Before medication is dispensed from the reservoir, the automated medication adherence system may be homed 1720. This may be accomplished by rotating the components of the automated medication adherence system until the homing transition of a homing strip is detected by an optical sensor. At the home position both the stage 1 and stage 2 gates may be closed. From this home position, opening the stage 1 gate may be accomplished, in one embodiment, by moving an inner cylinder in a counterclockwise direction and opening the stage 2 gate may be accomplished by moving the inner cylinder in clockwise direction from the home position. In this embodiment, the homing strip may be affixed to the inner cylinder. When switching between stage 1 and stage 2 configurations, the inner cylinder may pass through the home position allowing for the system to ‘re-home’ itself every transition for improved positional accuracy.

To start the dispense process, the gate of stage 1 may be opened by rotating the internal cylinder so that the two parabolic shaped cut-outs/openings in the inner and outer cylinder overlap to form a through-hole sized appropriately for the pill size and geometry 1730. This requires the system to know and be programmed with the size and shape of the pill 1710 that is loaded into the reservoir. The opening of the stage 1 gate may be accomplished in three (3) steps. First, the internal cylinder and outer cylinder may be rotated relative to one another (or one rotated while the other is static) in order to create a through-hole just below the minimum size for a pill to be dispensed.

At this point, the camera may monitor the stage 1 gate and the agitator may begin agitating 1733. Once the agitator begins agitating, the gate may continue to open at a slow rate up to a calculated maximum opening size 1735. Gate positions may be calculated based on pill size and geometry. The maximum opening size is large enough to allow one and only one pill to exit without allowing multiple pills to escape

Then, a pill may be dispensed from the stage 1 gate and detected via an optical sensor, light curtain, or other sensor mechanism, which may cause a signal to be sent to the control system to immediately close the gate and stop the agitator 1740. This effectively prevents another pill from passing through the stage 1 gate.

The pill may then be transferred from stage 1 to stage 2 1750. Although relatively rare, it is technically possible that two (2) or more pills exit stage 1 before the gate is closed. It is also possible, though even more rare, that two pills end up on top of each other in stage 2.

To account for accidental dispensing of an additional pill, the system may be configured to automatically activate the agitator with relatively high acceleration so that the pills in stage 2 end up in single file resting against the inner cylinder wall cylinder along the bottom of the agitator 1760. The system recognizes that stage 2 has more than one pill because the sensors captured the additional pill(s) exiting stage 1.

The gate of stage 2 may then be opened by rotating the internal cylinder in the opposite direction as compared to the stage 1 process, creating an appropriately sized through-hole by overlapping the inner and outer cylinder openings 1770.

Once the stage 2 gate is opened to the appropriate size for the pill size and geometry, the agitator may begin moving slowly in a single direction to drag the pill(s) towards the stage 2 gate through-hole 1780.

After a pill is detected as being dispensed from the stage 2 gate, the stage 2 gate is closed and at the same time the agitator may move in the opposite direction for a short distance to prevent any additional pills from being dispensed from stage 2 before the stage 2 gate is completely closed 1790.

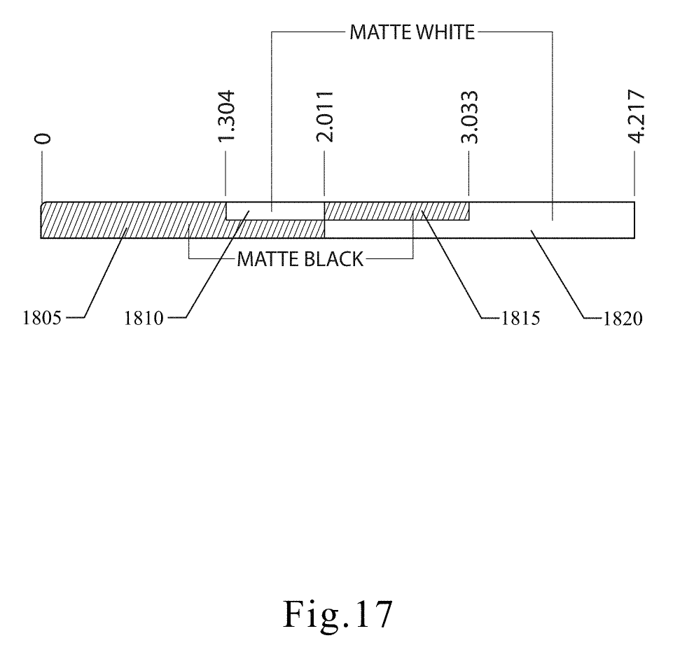

FIG. 17 is an illustration of a homing strip. As shown in FIG. 17, the homing strip 1800 may comprise four distinct sections, a first section 1805, a second section 1810, a third section 1815, and a fourth section 1820. Each of the four distinct sections may be visually distinct from one another. For example, the first section 1805 may be all black, the second section 1810 may be white on top and black on bottom, the third section 1815 may be black on top and white on bottom, and the fourth section 1820 may be all white. The homing strip 1800 may be affixed to a rotatable portion of an automated medication adherence system. An optical sensor configured to remain stationary as the homing strip moves may be utilized to determine the rotational configuration of the automated medication adherence system. For example, when an optical sensor has the intersection of the second and third sections 1810, 1815 at a center of its viewing window, the optical sensor may relay this information to a control system that may interpret this information to mean that the automated medication adherence system is at a “home” configuration, wherein no medications are being transferred from one stage to another. In a preferred embodiment, the automated medication adherence system “re-homes” before and/or after causing medication to be transferred from one area to another to ensure precision movement.

The width or size of the sections of the homing strip 1800 may each be different. In an alternate embodiment, the homing strip may comprise magnetic indicators.

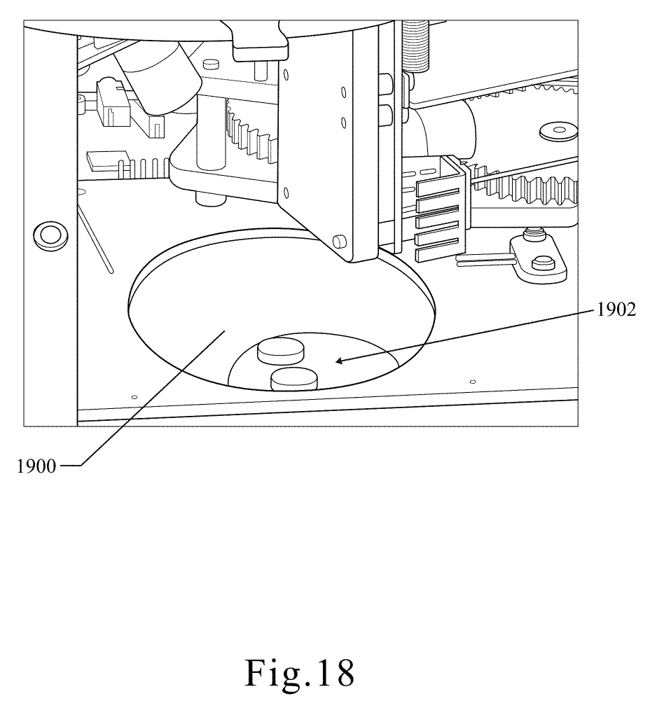

FIG. 18 is an illustration of one embodiment of the dispensing tray showing that pills have been dispensed into the dispensing tray. The dispensing tray 1900 is preferably configured to hold multiple pills 1902.

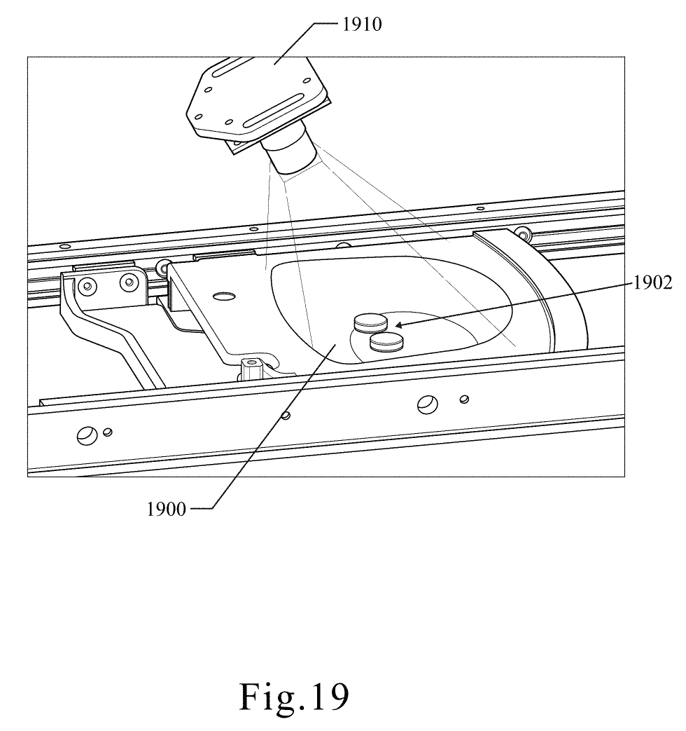

FIG. 19 is an illustration of one embodiment of the dispensing tray showing that a sensor determines whether the pills have been dispensed into the dispensing tray. The dispensing tray 1900 may be configured to be exposed to sensor 1910, which may determine the presence and or number of pills 1902 that are in the dispensing tray 1900.

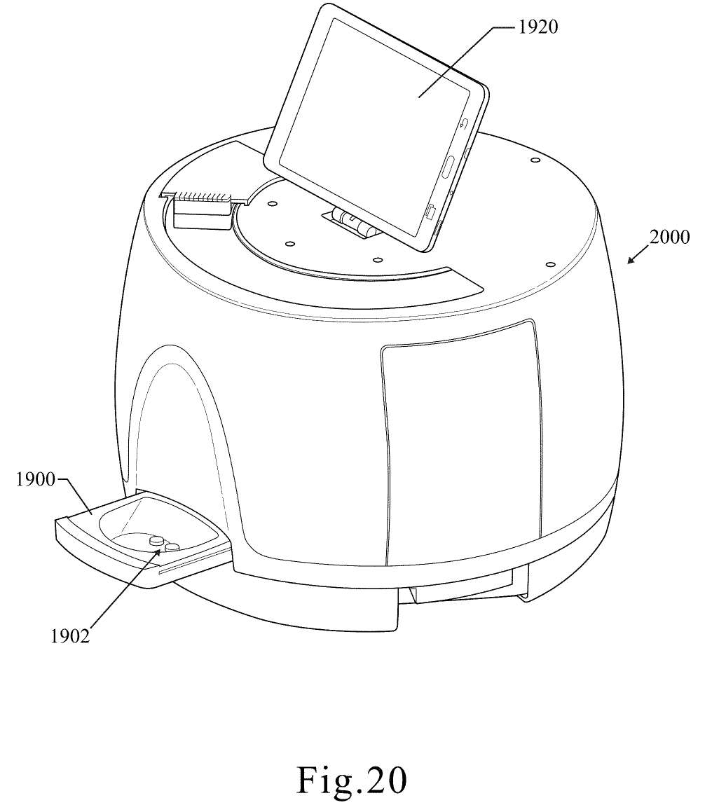

FIG. 20 is an illustration of one embodiment of the automated medication adherence system with the dispensing tray providing access to the pills. The automated medication adherence system 2000 may comprise a dispensing tray 1900 and interface 1920. The tray allows access to only those pills 1902 that a user is supposed to have at that time.

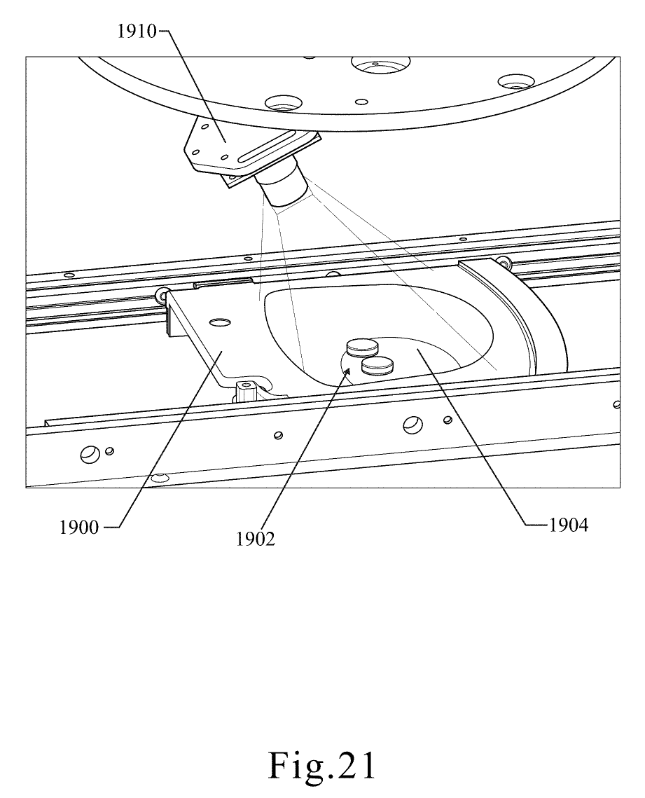

FIG. 21 is an illustration of one embodiment of the dispensing tray showing that pills were not removed from the dispensing tray. FIG. 21 shows the dispensing tray 1900 has been retracted into the automated medication adherence system, but some or all of the pills 1902 have not been removed by the user. The sensor 1910 senses that not all of the pills 1902 have been removed by the user. The sensor 1910 can determine how many and even what type of pills were not taken. The system saves information related to this failure of adherence. In order to remove the pills 1902, the tray floor 1904 may be moved or slid out of the way so that the pills 1902 drop out of the dispensing tray 1900.

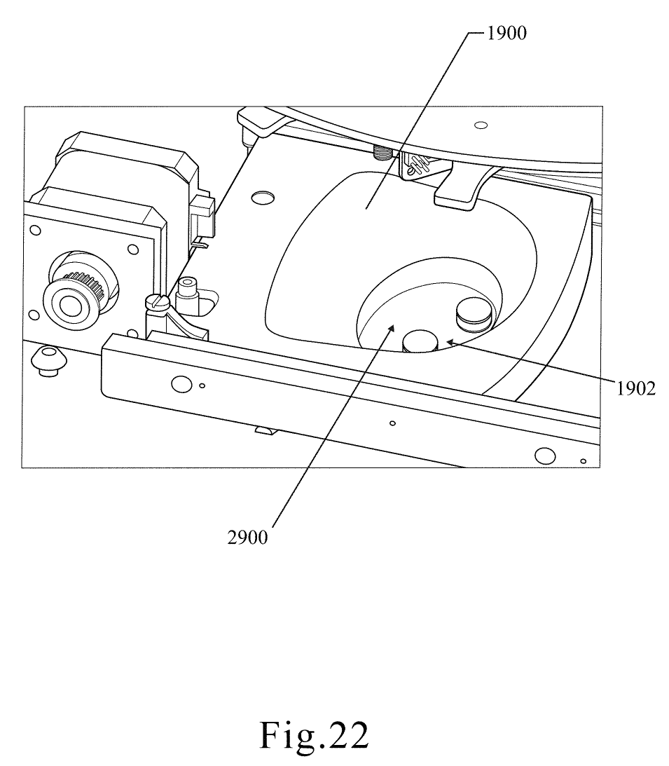

FIG. 22 is an illustration of one embodiment of the dispensing tray showing that pills can be removed from the dispensing tray by the automated medication adherence system. The floor of the dispensing tray 1900 may move so that the pills drop down into a holding area 2900. In this manner the pills 1902, which were not appropriately removed and taken by the user are not available or in the way the next time the system is use, and the system keeps the pills for future disposal.

Unless otherwise stated, all measurements, values, ratings, positions, magnitudes, sizes, locations, and other specifications that are set forth in this specification, including in the claims that follow, are approximate, not exact. They are intended to have a reasonable range that is consistent with the functions to which they relate and with what is customary in the art to which they pertain.

The foregoing description of the preferred embodiment has been presented for the purposes of illustration and description. While multiple embodiments are disclosed, still other embodiments will become apparent to those skilled in the art from the above detailed description. The disclosed embodiments capable of modifications in various obvious aspects, all without departing from the spirit and scope of the protection. Accordingly, the detailed description is to be regarded as illustrative in nature and not restrictive. Also, although not explicitly recited, one or more embodiments may be practiced in combination or conjunction with one another. Furthermore, the reference or non-reference to a particular embodiment shall not be interpreted to limit the scope. It is intended that the scope or protection not be limited by this detailed description, but by the claims and the equivalents to the claims that are appended hereto.

Unless otherwise stated, all measurements, values, ratings, positions, magnitudes, sizes, locations, and other specifications that are set forth in this specification, including in the claims that follow, are approximate, not exact. They are intended to have a reasonable range that is consistent with the functions to which they relate and with what is customary in the art to which they pertain.

The foregoing description of the preferred embodiment has been presented for the purposes of illustration and description. While multiple embodiments are disclosed, still other embodiments will become apparent to those skilled in the art from the above detailed description. These embodiments are capable of modifications in various obvious aspects, all without departing from the spirit and scope of protection. Accordingly, the detailed description is to be regarded as illustrative in nature and not restrictive. Also, although not explicitly recited, one or more embodiments may be practiced in combination or conjunction with one another. Furthermore, the reference or non-reference to a particular embodiment shall not be interpreted to limit the scope of protection. It is intended that the scope of protection not be limited by this detailed description, but by the claims and the equivalents to the claims that are appended hereto.

Except as stated immediately above, nothing that has been stated or illustrated is intended or should be interpreted to cause a dedication of any component, step, feature, object, benefit, advantage, or equivalent, to the public, regardless of whether it is or is not recited in the claims.

What is claimed is:

1. An automated medication adherence system, comprising:
a housing;
one or more sensors;
computing components; and
a plurality of reservoirs;
wherein said housing comprises a pill delivery and lock-out module and a carrier;
wherein said housing is configured to contain said plurality of reservoirs, which are mounted in said carrier;
wherein said carrier, said one or more sensors, and said computing components cause said plurality of reservoirs to move based on input from said computing components;
wherein at least one of said one or more sensors is a magnetic or optical sensor that monitors said carrier and control its position;
wherein said plurality of reservoirs are configured for receiving, storing, and dispensing a plurality of pills;
wherein said housing comprises an access cover;
wherein said housing comprises a reservoir loading door;
wherein said access cover comprises a pill loading assembly;
wherein said pill loading assembly is configured to allow said plurality of pills to be loaded into at least one of said plurality of reservoirs;
wherein said reservoir loading door comprises a reservoir loading assembly;
wherein said reservoir loading door is configured to allow said one or more reservoirs to be loaded and locked into said carrier;
wherein each of said plurality of reservoirs is configured to receive, store and dispense a homogenous type of pills of said plurality of pills;
wherein each of said plurality of reservoirs comprises a first stage, a second stage, a central agitation stalk, an outer wall cylinder, an inner wall cylinder, a homing strip, and one or more actuators;
wherein said outer wall cylinder and said inner wall cylinder, respectively, each have an opening at said first stage and an opening at said second stage;
wherein said central agitation stalk is configured to be substantially contained within said inner wall cylinder, and wherein said inner wall cylinder is configured to be substantially contained within said outer wall cylinder;
wherein said central agitation stalk is configured to be rotatable within said inner wall cylinder;
wherein said one or more sensors further comprise one or more optical sensors, such that at least one of said one or more optical sensors collects data regarding a rotational location of said homing strip, wherein said rotational location of said homing strip is usable to command the rotation of said inner wall cylinder and/or said outer wall cylinder to create a through-hole by overlapping one of said openings of the inner wall cylinder with one of said openings of said outer wall cylinder;
wherein said central agitation stalk comprises a wave surface, and a ribbed cone surface;
wherein said wave surface is a base of said first stage;
wherein said ribbed cone surface is a base of said second stage; and
wherein said wave surface is configured to cause said pills to separate and avoid clumping and travel from said first stage to said second stage and said ribbed cone surface is configured to cause said pills to travel from said second stage to said pill delivery and lock-out module.

2. The automated medication adherence system of claim 1, wherein said one or more sensors are interconnected via said computing components.

3. The automated medication adherence system of claim 1, wherein said one or more sensors are configured to determine when a single pill passes through each of said two successive stages.

4. The automated medication adherence system of claim 1, wherein at least one of said one or more sensors is selected from the group of sensors consisting of a magnetic sensor, an imaging sensor, a light curtain sensor, and combinations thereof.

5. The automated medication adherence system of claim 1, wherein said carrier moves rotationally, such that said plurality of reservoirs are configured to rotate within said housing.

6. The automated medication adherence system of claim 1, wherein said homing strip comprises an optical pattern.

7. The automated medication adherence system of claim 1, wherein said homing strip comprises a magnetic target.

8. The automated medication adherence system of claim 2, wherein said computing components cause said plurality of reservoirs mounted in said carrier to move in response to data regarding the location of each of said plurality of reservoirs as established by said sensor that monitors said carrier and control the position of said carrier.

9. The automated medication adherence system of claim 1, wherein data relating to a plurality of pill types is used to determine appropriate settings for dispensing said plurality of pill types.

10. The automated medication adherence system of claim 9, wherein said data relating to said plurality of pill types is selected from the group of data consisting of: a pill identity; a user identity; a dosage schedule; a medication format; a pill image; a pill geometry and size; a plurality of pharmaceutical indications for use; instructions for use; a physical description; a chemical description; a refill information; and a plurality of side effects information.

11. The automated medication adherence system of claim 2, wherein said first stage and said second stage are configured to be stacked, such that said first stage is physically above said second stage.

12. The automated medication adherence system of claim 11, wherein said first through-hole and said second through-hole are continuously variable in size by rotation of the inner wall cylinder in relation to the outer wall cylinder, such that said first through-hole and said second through-hole are both optimized for a specific pill type in a specific reservoir allowing said plurality of reservoirs to be identical and not dependent on which of said plurality of pill types is loaded in said specific reservoir

13. The automated medication adherence system of claim 11, wherein said first through-hole and said second through-hole are selectively openable and closable in response to said computing component;
wherein each of said first through-hole and said second through-hole, when open, allows one single pill at a time to pass through.

14. An automated medication adherence system, comprising:
one or more reservoirs;
a dispensing tray; and
a plurality of sensors, comprising at least a first through-hole sensor, a second through-hole sensor, and a dispensing tray sensor;
wherein each of said one or more reservoirs comprises two vertically stacked successive stages, an upper first stage and a lower second stage, a central agitation stalk, an outer wall cylinder, an inner wall cylinder, and one or more actuators;
wherein each of said one or more reservoirs is configured for receiving, storing, and dispensing a plurality of pills;
wherein said central agitation stalk is configured to be substantially contained within said inner wall cylinder, and wherein said inner wall cylinder is configured to be substantially contained within said outer wall cylinder;
wherein said central agitation stalk is configured to be rotatable within said inner wall cylinder;
wherein said central agitation stalk comprises a wave surface and a ribbed cone surface;
wherein said wave surface is a base of said first stage;
wherein said ribbed cone surface is a base of said second stage;
wherein dispensing of said plurality of pills by each of said one or more reservoirs are controlled by an electronic interface based on data gathered by said plurality of sensors;
wherein one or more of said plurality of pills are transferred from said first stage to said second stage one pill at a time;
wherein said one or more of said plurality of pills that are transferred to said second stage are transferred from said second stage to said dispenser tray one pill at a time.

15. The automated medication adherence system of claim 14, wherein each of said first stage and said second stage comprises a through-hole, such that there are two through-holes, a first through-hole and a second through-hole;
wherein said first through-hole sensor determines when a single pill of said plurality of pills passes through said first through-hole; and
wherein said second through-hole sensor determines when a single pill of said plurality of pills passes through said second through-hole.

16. The automated medication adherence system of claim 15, wherein said first opening and second opening are selectively openable and closable in response to said electronic interface.

17. The automated medication adherence system of claim 16, wherein said first opening and said second opening are variable in size such that rotation of said inner wall cylinder and said outer wall cylinder with respect to each other allows dispensing of pills of different sizes through said first through-hole and said second through-hole.

18. The automated medication adherence system of claim 17, wherein said one or more of said plurality of pills transferred to said tray dispenser from said second stage are sensed by said dispenser tray sensor before being dispensed.

19. The automated medication adherence system of claim 18, wherein said dispenser tray sensor determines whether said one or more of said plurality of pills dispensed in said tray dispenser are removed by a user;
wherein all of said plurality of pills not removed by a user from said dispenser tray are removed by said system from said dispenser tray to prevent over dosing.

20. An automated medication adherence system, comprising:
one or more reservoirs;
a dispensing tray; and
a plurality of sensors, comprising at least a first through-hole sensor, a second through-hole sensor, and a dispensing tray sensor;
wherein each of said one or more reservoirs comprises two vertically stacked successive stages, an upper first stage and a lower second stage, a central agitation stalk, an outer wall cylinder, an inner wall cylinder, and one or more actuators;
wherein each of said one or more reservoirs is configured for receiving, storing, and dispensing a plurality of pills;
wherein said central agitation stalk is configured to be substantially contained within said inner wall cylinder, and wherein said inner wall cylinder is configured to be substantially contained within said outer wall cylinder;
wherein said central agitation stalk is configured to be rotatable within said inner wall cylinder;
wherein said central agitation stalk comprises a wave surface, and a ribbed cone surface;
wherein said wave surface is a base of said first stage;
wherein said ribbed cone surface is a base of said second stage;
wherein dispensing of said plurality of pills by each of said one or more reservoirs is controlled by an electronic interface based on data gathered by said plurality of sensors;
wherein one or more of said plurality of pills are transferred from said first stage to said second stage one pill at a time;
wherein said one or more of said plurality of pills that are transferred to said second stage are transferred from said second stage to said dispenser tray one pill at a time;
wherein said one or more actuators are configured to rotate and agitate said central agitation stalk and rotate at least one of said inner wall cylinder and said outer wall cylinder, such that said inner wall cylinder and said outer wall cylinder are rotated with respect to each other;
wherein said outer wall cylinder comprises one or more outer wall cylinder openings and one or more chutes;
wherein said inner wall cylinder comprises one or more inner wall cylinder openings;
wherein when said inner wall cylinder and said outer wall cylinder are rotated with respect to each other, said one or more outer wall cylinder openings and said one or more inner wall cylinder openings align to form said two through-holes, a first through-hole and a second through-hole;
wherein said electronic interface comprises one or more logic algorithms;
wherein said one or more sensors, said one or more actuators, and said one or more logic algorithms are configured to control said inner wall cylinder, said outer wall cylinder, and said central agitation stalk to ensure that said plurality of pills are transferred, one pill at a time, from said first stage to said second stage; and
wherein said one or more sensors, said one or more actuators, and said one or more logic algorithms are configured to control said inner wall cylinder, said outer wall cylinder, and said central agitation stalk to ensure that said plurality of pills are transferred, one pill at a time, from said second stage to said dispenser tray.
